SEQUENCE LISTING (26 sequences)

SEQ ID NO: 1 - protein, 7 aa, synthetic construct
  REGION 1..7: note = Matrix-metalloprotease-responsive peptide sequence
PQGIWGQ

SEQ ID NO: 2 - protein, 8 aa, synthetic construct
  REGION 1..8: note = Matrix-metalloprotease-responsive peptide sequence
IPVSLRSG

SEQ ID NO: 3 - protein, 8 aa, synthetic construct
  REGION 1..8: note = Matrix-metalloprotease-responsive peptide sequence
VPMSMRGG

SEQ ID NO: 4 - protein, 8 aa, synthetic construct
  REGION 1..8: note = Cathepsin-responsive peptide sequence
VPMSMRGG

SEQ ID NO: 5 - protein, 4 aa, synthetic construct
  REGION 1..4: note = Elastase-responsive peptide sequence
AAPV

SEQ ID NO: 6 - protein, 9 aa, synthetic construct
  REGION 1..9: note = Elastase-responsive peptide sequence
APEEIMDRQ

SEQ ID NO: 7 - protein, 11 aa, synthetic construct
  REGION 1..11: note = Thrombin-responsive peptide sequence
  MOD_RES 4: note = Xaa = pipecolic acid
GGFXRYSWGCG

SEQ ID NO: 8 - protein, 10 aa, synthetic construct
  REGION 1..10: note = Thrombin-responsive peptide sequence
  MOD_RES 3: note = Xaa = Cyclohexylalanine
GGXARSWGCG

SEQ ID NO: 9 - protein, 12 aa, synthetic construct
  REGION 1..12: note = FXa-responsive peptide sequence
GGIEGRMGGWCG

SEQ ID NO: 10 - protein, 13 aa, synthetic construct
  REGION 1..13: note = Kallikrein-responsive peptide sequence
CGGGPFRIGGWCG

SEQ ID NO: 11 - protein, 6 aa, synthetic construct
  REGION 1..6: note = Aureolysin-responsive peptide sequence
ADVFEA

SEQ ID NO: 12 - protein, 5 aa, synthetic construct
  REGION 1..5: note = Aureolysin-responsive peptide sequence
AAEAA

SEQ ID NO: 13 - protein, 20 aa, synthetic construct
  REGION 1..20: note = Protease IV-responsive peptide sequence
MKATKLVLGAVILGSTLLAG

SEQ ID NO: 14 - protein, 9 aa, synthetic construct
  REGION 1..9: note = cell-instructive peptide sequence
KCWGRGDSP

SEQ ID NO: 15 - protein, 12 aa, synthetic construct
  REGION 1..12: note = cell-instructive peptide sequence
KCWGEIDGIELT

SEQ ID NO: 16 - protein, 9 aa, synthetic construct
  REGION 1..9: note = cell-instructive peptide sequence
KCWGIKLLI

SEQ ID NO: 17 - protein, 16 aa, synthetic construct
  REGION 1..16: note = cell-instructive peptide sequence
KGCWGGRNIAEIIKDI

SEQ ID NO: 18 - protein, 20 aa, synthetic construct
  REGION 1..20: note = cell-instructive peptide sequence
KGCWGGSDPGYIGSRSDDSA

SEQ ID NO: 19 - protein, 18 aa, synthetic construct
  REGION 1..18: note = cell-instructive peptide sequence
KGCWGGPQVTRGDVFTMP

SEQ ID NO: 20 - protein, 21 aa, synthetic construct
  REGION 1..21: note = cell-instructive peptide sequence
KGCWGGKGGNGEPRGDTYRAY

SEQ ID NO: 21 - protein, 8 aa, synthetic construct
  REGION 1..8: note = cell-instructive peptide sequence
CWGRGDSP

SEQ ID NO: 22 - protein, 7 aa, synthetic construct
  REGION 1..7: note = Adhesion peptide sequence
CWRGDSP

SEQ ID NO: 23 - protein, 15 aa, synthetic construct
  REGION 1..15: note = Matrix-metalloprotease-responsive peptide sequence
GCGGPQGIWGQGGCG

SEQ ID NO: 24 - protein, 9 aa, synthetic construct
  REGION 1..9: note = RGD-peptide sequence
GWGGRGDSP

SEQ ID NO: 25 - protein, 5 aa, synthetic construct
  REGION 1..5: note = RGD-peptide sequence
RGDSP

SEQ ID NO: 26 - protein, 10 aa, synthetic construct
  REGION 1..10: note = RGD-peptide sequence
CGWGGRGDSP

(19) United States
(12) Patent Application Publication
(10) Pub. No.: US 2025/0066562 A1
(43) Pub. Date: Feb. 27, 2025

(54) METHOD AND MATERIAL FOR DIFFERENTIATED SEQUESTRATION OF SUBSTANCES OF DIFFERENT SUBSTANCE GROUPS WITH THE AID OF HYDROGELS CONTAINING SULPHATED OR SULPHONATED COMPONENTS

(71) Applicant: LEIBNIZ-INSTITUT FÜR POLYMERFORSCHUNG E.V., Dresden (DE)

(72) Inventors: Lucas Schirmer, Dresden (DE); Uwe Freudenberg, Dresden (DE); Carsten Werner, Dresden (DE); Passant Attalah, Dresden (DE)

(73) Assignee: LEIBNIZ-INSTITUT FÜR POLYMERFORSCHUNG E.V., Dresden (DE)

(21) Appl. No.: 18/913,092

(22) Filed: Oct. 11, 2024

Related U.S. Application Data
(62) Division of application No. 16/492,238, filed on Sep. 9, 2019, now abandoned.

(30) Foreign Application Priority Data
Mar. 10, 2017 (DE) 10 2017 105 195.3

(51) Int. Cl. C08J 3/24; A61K 9/06; A61P 31/04; C08B 37/00; C08L 5/10; C12N 5/00; C12N 5/073; C12N 5/074; C12N 5/0775

(52) U.S. Cl. CPC C08J 3/24; A61K 9/06; A61P 31/04; C08B 37/0075; C08L 5/10; C12N 5/0018; C12N 5/0605; C12N 5/0668; C12N 5/0696; C08J 2325/14; C08L 2203/02; C12N 2501/115; C12N 2501/165; C12N 2501/999; C12N 2533/30; C12N 2533/70; C12N 2537/10

(57) ABSTRACT

A method is disclosed for differentiated sequestration of substances of different substance groups A and B in a sulfated and/or sulfonated hydrogel while simultaneously releasing substances of substance group A or B from the sulfated and/or sulfonated hydrogel into a biofluid. The sulfated and/or sulfonated hydrogel is selected from hydrogels of Type 1, Type 2, Type 3, Type 4 and consist of uncharged and charged components. The charged components are characterized by calculating the number of sulfated or sulfonated groups per repeat unit divided by the molecular mass of the repeat unit, for each of Type 1, 2, 3 and 4. Swollen hydrogels have a different concentration of sulfated or sulfonated groups in mmol/ml for Type 1, Type 2, Type 3 and Type 4. The concentration of substances of each substance group A and group B in the biofluid is influenced by the selection of the type of hydrogel.

### CROSS-REFERENCES TO RELATED APPLICATIONS

This application is a divisional of pending U.S. application Ser. No.: 16/492,238 which is a U.S. National Stage of International Application No. PCT/DE2018/100218, filed Mar. 9, 2018, which designated the United States and has been published as International Publication No. WO 2018/162009 A2 and which claims the priority of German Patent Application, Serial No. 10 2017 105 195.3, filed Mar. 10, 2017, pursuant to 35 U.S.C. 119(a)-(d) the descriptions of which are hereby incorporated by reference.

### REFERENCE TO AN ELECTRONIC SEQUENCE LISTING

The contents of the electronic sequence listing, Schirmer5-3.xml: size 34,743 bytes, created on Nov. 19, 2024, is herein incorporated by reference in its entirety.

### BACKGROUND OF THE INVENTION

The invention relates to a method for differentiated sequestration of substances of different substance groups A and B with the aid of hydrogels containing sulfated and/or sulfonated components and the depletion of the substances of substance group A from a biofluid with simultaneous differentiated release into the biofluid of substances of substance group A or B from the hydrogel containing sulfated and/or sulfonated components and/or the reduced binding of the substances of substance group B in the hydrogel containing sulfated or sulfonated component. The invention further relates to hydrogels which can be synthetically obtained based on poly (4-styrenesulfonic acid-co-maleic acid) as a network component and amine-or thiol-containing crosslinking molecules as another network component, and which can be characterized and used for the above method as a material for the differentiated sequestration of substances of different groups of substances. The substance groups A and B are not chemically defined substance groups, but may contain, depending on the type of hydrogel, different substances which cause in the corresponding constellation and composition the differentiated sequestration according to the invention within the overall system. The substance groups A and B are thus defined by their behavior in the overall system.

The field of application of the invention is in the field of biotechnology and medicine, where certain substances are selectively removed from a biofluid at a molecular level and sequestrated in a hydrogel and where, on the other hand, other substances are not specifically sequestered but are selectively released from the hydrogel into the biofluid. Thus, in a broader sense, it is a molecular-level separation process. In a broader sense, the field of application of the invention lies in the use of graded negatively charged hydrogels for technical, biomedical and biological applications, for example for the cultivation of mammalian cells or an antibacterial finish of surfaces.

Hydrogels with sulfated components, for example star-PEG-glycosaminocyclo-hydrogels, are known in the art from WO 2010/060485 A1 and are being investigated for use in biotechnological applications or as implant or tissue replacement materials for use in regenerative therapies.

A key feature of these materials lies in resulting interactions between sulfate groups on polymer chains of a component of the hydrogels with soluble proteins, such as for example proteins controlling the metabolism, transport and signaling functions, such as enzymes and signaling molecules, wherein the former include for example, proteases, lipolases, amylases and the latter include, for example, hormones, neurotransmitters, cytokines, growth factors and chemokines.

The interactions crucial for the affinity to these proteins are based primarily on electrostatic forces between the sulfate or sulfonic acid groups of the hydrogels which are negatively charged under application conditions and amino acid side chains of proteins which are positively charged.

This principle is already widely used in the art for the development of hydrogels containing sulfated sugars (glycosaminoglycans=GAGs) for sustained release of signaling molecules, for example for the growth factors VEGF (Vascular Endothelial Growth Factor) and FGF-2 (Fibroblast Growth Factor 2). In addition, the release of FGF-2 and VEGF could be gradually increased by reducing the degree of sulfation in the underlying star-PEG-GAG hydrogels. Synthetic polymers containing sulfonic acid groups, for example, polystyrene sulfonic acid (PSS), J. Phys. Chem. B, 2007, 111 (13), pp 3391-3397, DOI: 10.1021/jp067707d9., or 2-acrylamido-2-methylpropanesulfonic acid (U.S. Pat. Nos. 5,451,617 and 5,011,275 and US Patent Application No. 2008/011412) have been used as a sulfonated component to produce hydrogels, for example for use with contact lenses.

However, there are currently no known covalently crosslinked, hydrous polymer networks carrying sulfonic acid groups, i.e., hydrogels, in which the carboxyl groups of poly (4-styrenesulfonic acid-co-maleic acid) have been used as functional groups for crosslinking with amine-group-containing short crosslinkers or amine-group-containing polymers.

Another disadvantage is that the interactions between hydrogels containing sulfated or sulfonated components (or building blocks), and proteins have thus far been studied mostly only from solutions with one to two signaling molecules and frequently not under physiologically relevant conditions. However, in the relevant biological context, biofluids with a variety of different proteins are present at different concentrations, with the signaling molecules being present in biofluids only at low levels in the 100 ng/ml-2000 ng/ml range, while other proteins such as albumin are present in high concentrations of about 60 mg/ml.

Here, the primary electrostatic attractive forces between positively charged domains of signaling molecules and the negatively charged sulfate or sulfonate groups in the hydrogel play an important role, wherein the isoelectric point (IEP) of a protein can often be used for their estimation. In addition to the net charge under physiological conditions which can be estimated on the basis of the IEP, however, the distribution of the charge, for example the presence of positively charged charge clusters, as well as secondary interactions, which are influenced by protein size and protein structure and by the formation of weaker, nonionic, intermolecular forces, such as, for example, hydrophobic interactions, hydrogen bonds or dipole interactions, play a role, so that it has thus far not been possible to predict the absolute or relative binding of the proteins to the hydrogels. In addition, there are additional competing processes that have often been neglected, for example due to interactions of the target proteins with albumin, which is responsible for the control of osmotic pressure and the regulation of transport processes, as well as interactions between albumin with the hydrogel. Furthermore, the binding constants used in the literature for predicting the molecular interactions between charged polymers and proteins are only suitable for describing the interaction of individual molecule-protein complexes in solution, whereas in the hydrogels the adjacent polymer chains and the three-dimensional distribution of the charge or affinity centers in the polymer network have a strong influence on the resulting interactions and thus on the binding of proteins in the hydrogel.

Accordingly, the selectivity of binding between hydrogels containing sulfated and/or sulfonated components (building blocks) and molecular components of complex biofluids could hitherto not be elucidated. Accordingly, the established methods and processes are able only to a limited extent to selectively control the levels of signaling molecules by hydrogels containing sulfated or sulfonated components (building blocks) in application-relevant biofluids.

### SUMMARY OF THE INVENTION

It is one aspect of the invention to propose a method which allows intentionally influencing the concentration of certain substances in biofluids.

This aspect and others are achieved by a method, wherein the method includes differentiated sequestration of substances of different substance groups A and B in a sulfated and/or sulfonated hydrogel and for depletion of substances of substance group A from a biofluid with simultaneous differentiated release of substances of substance group A or B from the sulfated and/or sulfonated hydrogel into the biofluid or the reduced binding of substances of the substance group B in the sulfated and/or sulfonated hydrogel, wherein the sulfated and/or sulfonated hydrogel is selected from a group of type 1, type 2, type 3 or type 4 hydrogels and the hydrogels are composed of uncharged building blocks and charged building blocks are characterized by a parameter calculated from the number of sulfate and/or sulfonate group per repeat unit divided by the molar mass of the repeat unit of 0.0040-0.0060 mole/g for type 1, of 0.0025-0.0040 mole/g for type 2, of 0.0005-0.0025 mole/g for type 3, and of 0040 to 0.0100 mole/g for type 4 and the swollen hydrogels have a storage module of less than 20 kPa and the properties of the swollen hydrogels have a concentration of sulfate or sulfonate groups in mmole/ml between 0.09 to 0.20 in type 1, between 0.05 to 0.18 for type 2, between 0.01 to 0.12 for type 3, and between 0.16 to 0.8 for type 4, and that an influence on the concentration of substances of substance group A and of substances of substance group B in the biofluid is determined by the choice of the type of the hydrogel.

Other aspects become clear from the further description. As a material suitable for carrying out the process, a fully synthetic hydrogel system based on poly (4-styrenesulfonic acid co-maleic acid) was used as a synthetic component which is negatively charged under physiological conditions and hence affine for partially positively charged biomolecules for the differentiated sequestration of substances from various substance groups. Further developments of the method and the material are recited in the dependent claims. The term “fully synthetic” means that no components of biological origin are required for hydrogelation. Therefore, the risk of adverse immunogenic reactions can be excluded.

The sequestration of substances from the substance groups A and B in the context of the invention refers to the binding of these substances, such as signaling molecules, factors or enzymes, to affinity centers in a hydrogel material and thus reducing the concentration or the complete removal of these substances from a biofluid in direct contact with the hydrogel. The hydrogel consists of building blocks that are charged or uncharged.

The invention includes, as hydrogels containing sulfate and/or sulfonate groups, hydrogels having the following properties and the following composition: Polymeric networks formed by covalent (chemical) crosslinking or physical crosslinking, for example according to WO 2014040591 A2, between two hydrogel components or hydrogels building blocks. The first building block or component of the hydrogel is a molecule which is uncharged under physiological conditions, also referred to as uncharged building block (UGB), and preferably having a molar mass from 20 g/mole to 100,000 g/mole. The uncharged molecules are advantageously selected or derived from the class of polyethylene glycols, poly (2-oxazolines), polyvinyl pyrolidones (PVP), polyvinyl alcohols (PVA), and/or polyacrylamides (PAM) or a short bifunctional crosslinker molecule. Furthermore, the UGB has at least two functional groups, preferably 4 to 8 functional groups, which are particularly advantageous for crosslinking. Suitable functionalities for crosslinking (at the GB or UGB) may include amine, thiol, carboxyl, anhydride, maleimide, vinylsulfone, acrylate, hydroxyl, isocyanate, epoxide, and aldehyde groups, or groups capable of forming noncovalent bonds based on electrostatic forces, hydrophobic interactions, hydrogen bonds, dipole interactions. The second building block (component) consists of a polymer having (sulfurous) sulfate or sulfonate groups which is thus negatively charged under physiological conditions (charged building block, GB) (optionally having a molar mass of 2,000 to 250,000 g/mole) capable of binding proteins in the hydrogel network primarily via the electrostatic (ionic) interactions and, to a lesser extent, via weaker bonds such as van der Waals forces, hydrogen bonds or hydrophobic interactions. According to the invention, the affinity centers, which significantly determine the binding of proteins, are the sulfate or sulfonic acid groups which are largely negatively charged under physiological conditions. Sulfated or sulfonated polymers are sulfated glycosaminoglycans obtained from natural sources, such as heparin and selectively desulfated heparins, chondroitin sulfate, heparan sulfate, keratan sulfate, sulfated hyaluronic acid, as well as sulfated glycopolymers based on mannose, lactose, dextran and polysulfonated compounds which may have as sulfur-containing monomers, for example, styrenesulfonic acid (SS), vinylsulfonic acid (VS), 2-acrylamido-2-methylpropanesulfonic acid (AMPS), aminopropanesulfonic acid (APS) or anetholesulfonic acid (AS) (also in copolymers with units containing the aforementioned crosslinking groups).

The affinity centers, negatively charged sulfate or sulfonate groups, are intentionally distributed only along the GB, whereas the UGB is a component minimizing protein adsorption. Any carboxyl groups present on the GB and not used for the crosslinking reaction are considered to be irrelevant for the binding of the proteins in the hydrogel because of the significantly more acidic and therefore preferably deprotonated sulfate or sulfonate groups of the GB.

Physiological conditions are aqueous solutions which in tissue having a pH value and an ionic strength corresponding exactly to a physiological salinity for human tissue and are thus adjusted to pH 7.4 and about 0.9% NaCl with a phosphate buffer.

A biofluid according to the invention is an aqueous solution having a variable salt content, preferably a physiological salinity, and a mixture of proteins.

The protein content can vary and consists mostly of water-soluble globular proteins, for example signaling molecules, enzymes or factors, at low levels in a range of 100 pg/ml-2000 ng/ml and albumins which regulate the osmotic pressure and transport processes in the body and are present, for example, in high concentration of about 60 mg/ml.

According to the concept of the invention, differences of proteins controlling the binding of the metabolism, transport, and signal function (e.g. signaling molecules and enzymes) to sulfated or sulfonated components (GB) containing hydrogels are significantly determined, on the one hand by the electrostatic interactions between GB and proteins, as well as the size and the structure of the proteins (for example, the presence of characteristic protein domains) and, on the other hand by the network parameters of the hydrogels. As determining network parameters were measured: (1) the sulfate or sulfonate concentration in the swollen hydrogel (mmole sulfate or sulfonate per ml) in the hydrogel swollen under physiological conditions; and (2) the number of sulfate or sulfonate groups per repeat unit of the GB divided by the molar mass of the repetition units of the GB (number of groups/(g/mole)) as a characteristic size for the charge of the GB. The parameter (1) is correlated with the number of interaction centers (corresponding to negatively charged sulfate or sulfonate groups) in the three-dimensionally swollen hydrogel network and will therefore significantly determine the binding of proteins (signaling molecules, enzymes) in the hydrogel. This parameter includes intermolecular interactions (i.e., interactions of adjacent polymer chains) of the GB with proteins (signaling molecules, enzymes). The parameter (2), on the other hand, describes the density and distribution of the interaction centers (corresponding to negatively charged sulfate or sulfonate groups) along the different GBs and will thus determine the interactions of the proteins with the respective individual GBs and thus also includes specific interactions via the spatial modulation of the charge centers the polymer chain which are known, for example, for protein-glycosaminoglycan interactions (see Capila, I. Linhardt, RJ Angew Chem Int Ed Engl 2002, 41 (3), 391-412). The binding and release behavior of the hydrogels for proteins (signaling molecules) is determined according to the invention by a superimposition of the resulting hydrogel network properties resulting from both parameters (1) and (2) and can thus be quantitatively described by specifying these parameters (see also results for the exemplary embodiments UGB1-GB1 01 to UGB1-GB4 04).

In addition, steric effects must be considered because for binding and sequestration of proteins steric accessibility of the hydrogel network must be guaranteed, i.e. the size of the proteins must not exceed the mesh size of the hydrogels, for example, estimated from the storage modulus of the hydrogels by way of rubber elasticity theory with respect to material and methods.

By contacting biofluids with hydrogels which can be selectively graded in their network structure and their degree of sulfation or sulfonation, the concentration of relevant signaling molecules in the biofluid can be adjusted through a characteristic selectivity of the binding of signaling molecules to the hydrogel and can accordingly be controlled in a biological or biotechnological application. The biological application can in the medical context relate to the modulation of soluble signaling molecules for the control of angiogenesis, sprouting of blood vessels, the immune response, inter alia, for curing neurodegenerative and autoimmune and cancer diseases, diabetes, in cutaneous wound healing and bone regeneration, for inhibiting tumor proliferation and for antiseptic and antimicrobial treatment in or on the body. Biotechnological applications include in vitro cell and organ culture of embryonic stem cells(ES), induced pluripotent stem cells (iPS), and other non-ES-and iPS-associated stem and progenitor cells, primary, patient-derived cells, immortalized cell lines, as well as heart, muscle, kidney, liver and nerve tissue as well as in the enrichment or depletion and thus the separation of protein mixtures, in particular signaling molecule mixtures or enzyme mixtures.

Another aspect of the invention relates, as already mentioned, to a covalently crosslinked hydrogel material for carrying out the above method, based on charged building blocks in the form of poly (4-styrenesulfonic acid-co-maleic acid) and uncharged building blocks in the form of amine groups or thiol groups containing polymers or crosslinker molecules having at least two amino or thiol groups. The charged and uncharged building blocks are crosslinked to form a polymer network which can be obtained by activating the carboxyl groups of the poly (4-styrenesulfonic acid-co-maleic acid) with 1-ethyl-3-(3-dimethylaminopropyl)-carbodiimide (EDC)/N-hydroxysulfosuccinimide (sulfo-NHS) and either direct crosslinking with polymers containing the amine groups or the crosslinker molecules having the at least two amino groups each with amide formation or a functionalization of the activated carboxyl groups by means of bifunctional crosslinker molecules, each having an amino group and a group capable of Michael-type addition, and the subsequent crosslinking with the polymers containing thiol groups or the crosslinker molecules having the at least two thiol groups each via a respective Michael-type addition. The group capable of a Michael-type addition is preferably selected from maleimide, vinylsulfone and acrylate groups. The polymers containing amine or thiol groups as uncharged building blocks are preferably selected from the class of polyethylene glycols (PEG), poly (2-oxazolines) (POX), polyvinylpyrrolidone (PVP), polyvinyl alcohols (PVA) and/or polyacrylamides (PAM). The alternatively used amine or thiol groups containing short crosslinker molecules are preferably nonpolymeric bifunctional crosslinker molecules. Poly (4-styrenesulfonic acid-co-maleic acid) as a charged building block is advantageously selected with variable molar ratios of 4-styrenesulfonic acid to maleic acid in the range of 6:1 to 1:6 and molar masses in the range of 5,000 to 100,000 g/mole. According to a particularly preferred embodiment of the invention, polymers with enzymatically cleavable peptides for polymer network formation are used as uncharged building blocks. These enzymatically cleavable peptides preferably have as a reactive amino acid in the peptide sequence either lysine, with an amino group in the side chain, or cysteine, with a thiol group in the side chain. The enzymatically cleavable peptides are advantageously cleavable with the aid of human or bacterial proteases, in particular (MMPs)-responsive matrix metalloproteinases such as SEQ ID NO 1, SEQ ID NO 2, or SEQ ID NO 3, cathepsin-responsive such as SEQ ID NO 4, elastase-responsive such as SEQ ID NO 5 or SEQ ID NO 6, blood-clotting-enzyme-responsive such as thrombin-responsive GGF-pipecolic acid SEQ ID NO 7, or GG-cyclohexylalanine SEQ ID NO 8, FXa-responsive such as SEQ ID NO 9, calikrein-responsive such as SEQ ID NO 10, or bacterial-protease-responsive such as aureolysin-responsive SEQ ID NO 11, or SEQ ID NO 12, elastase-responsive AAPV or the protease IV-responsive sequence SEQ ID NO 13. Hydrogels constructed in this way allow autoregulative release and degradation mechanisms. According to another embodiment of the invention, bioactive and/or antiadhesive molecules having an amino or carboxyl group and/or cell-engineering peptides, in particular selected from SEQ ID NO 14, SEQ ID NO 15, SEQ ID NO 16, SEQ ID NO 17, SEQ ID NO 18, SEQ ID NO 19, SEQ ID NO 20 are attached via lysine or cysteine in the sequence to the charged building block poly (4-styrenesulfonic acid-co-maleic acid) or its derivatives with Michael-type addition-capable groups to the hydrogel network by forming a covalent bond. The bioactive molecules may be antimicrobial substances, for example antibiotics or antiseptics, or pharmaceutical agents. The anti-adhesive molecules are preferably polyethylene glycols (PEG) or poly (2-oxazolines) (POX). The cell-engineering peptides are preferably peptides derived from structural and functional proteins of the extracellular matrix, for example peptides derived from collagen, laminin, tenascin, fibronectin and vitronectin. According to an advantageous embodiment, the bioactive and/or antiadhesive and/or cell-engineering peptides are covalently coupled to the hydrogel networks via enzymatically cleavable peptide sequences. The enzymatically cleavable peptides are, as already mentioned, preferably sensitive to human or bacterial proteases, for example MMPs, cathepsins, elastases, blood coagulation enzymes. Hydrogels of this type allow autoregulative release and degradation mechanisms even while retaining the hydrogel network. Preferably, the hydrogel material has a storage modulus of 0.2 to 22 kPa. The sulfonate concentration in the swollen network and the number of sulfonate groups per repeat unit (WE) divided by the molar mass (MW) of the repeat unit in (g/mole) can be varied independently of one another according to the ranges defined in Table 5.1 (see below).

Thus, the invention provides a fully synthetic, highly hydrated hydrogel, which carries affinity centers for substance groups to be separated via the sulfonate groups of poly (4-styrenesulfonic acid-co-maleic acid) as a charged building block (GB), with intentionally gradable physical and biochemical properties. The poly (4-styrenesulfonic acid-co-maleic acid) is used here as a charged building block (GB) in the sense of the previously described hydrogel material. This material offers the possibility to map all important functions of the natural extracellular matrix (ECM) in a modular way, i.e. largely independently of one another. In detail, these functions are the framework, support and protection function for growing cells as the first function, the control of cell adhesion as the second function, the graded sequestration and reversible release of therapeutically relevant signaling molecules as the third function, and the possibility of on-demand reorganization by ingrowth cells as the fourth function. The physical properties, such as stiffness and hydration of the material, are adjustable over a wide range. According to a preferred embodiment of the invention, covalently crosslinked hydrogels were prepared for this purpose by reacting carboxyl groups of the poly (4-styrene sulfonic acid-co-maleic acid) activated with 1-ethyl-3-(3-dimethylaminopropyl)-carbodiimide (EDC)/N-hydroxysulfosuccinimide (sulfo-NHS) and terminal amino groups of a linear or star-branched polyethylene glycol as an uncharged building block via stable amide groups. In an alternative embodiment, which is preferred when embedding living cells, in a first step, the carboxyl groups poly (4-styrene sulfonic acid-co-maleic acid) activated with 1-ethyl-3-(3-dimethylaminopropyl)-carbodiimide (EDC)/N-hydroxysulfosuccinimide (sulfo-NHS) are graded by means of the short bifunctional crosslinker molecule N-(2-aminoethyl) maleimide, i.e. functionalized with 6 to 10 molecules of N-(2-aminoethyl) maleimide per molecule of poly (4-styrenesulfonic acid-co-maleic acid), and then purified and isolated. These derivatives of the poly (4-styrenesulfonic acid-co-maleic acid) with N-(2-aminoethyl) maleimide then allow the spontaneous, bio-orthogonal reaction when mixed in aqueous solutions or in a complex biofluid with living cells and biomolecules without their functional change via a Michael- type addition between the maleimide groups of the functionalized poly (4-styrenesulfonic acid-co-maleic acid) derivatives and the thiol groups of the thiol-group- containing bifunctional crosslinker molecules or polymers containing thiol groups as uncharged building blocks within the meaning of the previously described hydrogel structure. These building blocks may, for example, be enzymatically-cleavable peptides having in their sequence the amino acid cysteine (which has a thiol group in the side chain) and which has previously been conjugated to one or more arms of a four-arm polyethylene glycol (PEG), as described in Tsurkan et al., 2013 (Adv. Mater. 2013, 25, 2606-2610). This type of crosslinking has the advantage that due to the rapid reaction of the maleimides with the free thiol groups, a directed reaction occurs without unwanted side reactions with other biomolecules or with proteins of the cell surfaces, so that the Michael-type addition can be used as a bio-orthogonal crosslinking reaction for polymerization cells. The hydrogels can also be functionalized via the maleimide groups of the poly (4-styrenesulfonic acid-co-maleic acid) derivatives by means of cell- engineering peptides, for example the amino acid sequence SEQ ID NO 21. In addition, the hydrogel materials may be functionalized with antimicrobial substances, for example, with positively charged antibiotics.

As an alternative to the covalently crosslinked hydrogel material, a physically crosslinked hydrogel material may be applied for carrying out the above- mentioned method. This likewise fully synthetic material is based on physical interactions between charged building blocks in the form of poly (4-styrenesulfonic acid- co-maleic acid) with uncharged building blocks in the form of polymers, wherein highly positively charged peptide sequences are conjugated onto the polymers. The highly positively charged peptide sequences preferably comprise at least ten repeats of lysine or arginine or at least five repeats of dipeptide motifs with lysine and alanine or with arginine and alanine.

### BRIEF DESCRIPTION OF THE DRAWING

Further details, features and advantages of embodiments of the invention will become apparent from the following description of exemplary embodiments, which are also described and supported by the depicted tables and wherein the sole Figure shows representative light-microscopic images of cell cultures with human endothelial cells cultured on hydrogels after 24 hours.

### DETAILED DESCRIPTION OF PREFERRED EMBODIMENTS

Other advantageous concentration ratios can also be set by deliberately varying the network properties, and the proposed method also makes it possible to control the concentration of these species in any biofluid by extending the analysis to additional signaling molecules.

TABLE 1
Chemical and physical description of the hydrogels, embodiments 01 to 26: crosslinking reaction 1 (VN1): activated
carboxyl group with amino group, crosslinking reaction 2 (VN2): maleimide group with thiol group

| | Hydrogel mixture during | | | Hydrogel properties under physiological conditions, swollen | | | | | | |
|---|---|---|---|---|---|---|---|---|---|---|
| | crosslinking, unswollen | | | | | | Concen- | | | |
| | Concen- | Concen- | | | | Concen- | tration | Storage module | | |
| | tration | tration | Molar | Swelling | | tration | sulfate/ | in kPa | | |
| | UGB in | GB in | Ratio | Average | | GB in | sulfonate in | Average | | |
| Name | mmole/ml | mmole/ml | UGB:GB | Value | SD | mmole/ml | mmole/ml | value | SD | VN |
| UGB1-GB1 01 | 0.0091 | 0.0030 | 3 | 1.77 | 1.70 | 0.0018 | 0.12 | 1.9 | 1.9 | 1 |
| UGB1-GB2 02 | 0.0079 | 0.0040 | 2 | 1.62 | 0.03 | 0.0025 | 0.12 | 4.0 | 1.1 | 1 |
| UGB1-GB3 03 | 0.0073 | 0.0045 | 1.5 | 1.71 | 0.04 | 0.0027 | 0.06 | 2.3 | 0.0 | 1 |
| UGB1-GB4 04 | 0.0057 | 0.0038 | 1.5 | 1.23 | 0.01 | 0.0031 | 0.28 | 7.9 | 1.9 | 1 |
| UGB1-GB4 05 | 0.0079 | 0.0026 | 3 | 1.54 | 0.54 | 0.0017 | 0.15 | 14.5 | 1.6 | 1 |
| UGB1-GB4 06 | 0.0030 | 0.0020 | 1.5 | 1.54 | 0.05 | 0.0013 | 0.12 | 7.3 | 0.2 | 1 |
| UGB1-GB4 07 | 0.0041 | 0.0014 | 3 | 1.12 | 0.03 | 0.0012 | 0.11 | 13.7 | 0.5 | 1 |
| UGB1-GB4 08 | 0.0052 | 0.0009 | 6 | 0.95 | 0.03 | 0.0009 | 0.08 | 19.6 | 2.0 | 1 |
| UGB1-GB4 09 | 0.0021 | 0.0014 | 1.5 | 1.25 | 0.18 | 0.0011 | 0.1 | 4.8 | 0.6 | 1 |
| UGB1-GB4 10 | 0.0030 | 0.0010 | 3 | 1.01 | 0.12 | 0.0010 | 0.09 | 8.1 | 0.7 | 1 |
| UGB1-GB4 11 | 0.0037 | 0.0006 | 6 | 0.86 | 0.11 | 0.0007 | 0.06 | 8.5 | 0.4 | 1 |
| UGB1-GB5 12 | 0.0057 | 0.0038 | 1.5 | 1.68 | 35.00 | 0.0023 | 0.09 | 8.5 | 1.7 | 1 |
| UGB1-GB5 13 | 0.0079 | 0.0026 | 3 | 1.48 | 0.23 | 0.0018 | 0.13 | 15.3 | 0.7 | 1 |
| UGB1-GB5 14 | 0.0030 | 0.0020 | 1.5 | 1.81 | 0.04 | 0.0011 | 0.08 | 5.5 | 1.4 | 1 |
| UGB1-G85 15 | 0.0041 | 0.0014 | 3 | 1.28 | 0.08 | 0.0011 | 0.08 | 12.5 | 1.2 | 1 |
| UGB1-GB5 16 | 0.0052 | 0.0009 | 6 | 0.99 | 0.04 | 0.0009 | 0.06 | 21.8 | 0.8 | 1 |
| UGB1-GB5 17 | 0.0021 | 0.0014 | 1.5 | 1.58 | 0.08 | 0.0009 | 0.07 | 3.0 | 0.2 | 1 |
| UGB1-GB5 18 | 0.0030 | 0.0010 | 3 | 1.05 | 0.10 | 0.0009 | 0.07 | 6.9 | 0.7 | 1 |
| UGB1-GB5 19 | 0.0037 | 0.0006 | 6 | 0.90 | 0.04 | 0.0007 | 0.05 | 8.0 | 1.3 | 1 |
| UGB2-GB4 20 | 0.0015 | 0.0022 | 1.5 | 1.22 | 0.03 | 0.0018 | 0.16 | 3.7 | 0.5 | 2 |
| UGB2-GB4 21 | 0.0015 | 0.0015 | 1 | 1.62 | 0.20 | 0.0009 | 0.08 | 1.1 | 0.2 | 2 |
| UGB2-GB4 22 | 0.0015 | 0.0011 | 0.75 | 2.20 | 0.19 | 0.0005 | 0.05 | 0.2 | 0.1 | 2 |
| UGB2-GB4 23 | 0.0015 | 0.0022 | 1.5 | 1.19 | 0.17 | 0.0018 | 0.14 | 4.6 | 1.5 | 2 |
| UGB2-GB5 24 | 0.0015 | 0.0015 | 1 | 1.56 | 0.07 | 0.0010 | 0.07 | 1.2 | 0.2 | 2 |
| UGB2-GB5 25 | 0.0015 | 0.0011 | 0.75 | 1.95 | 0.72 | 0.0006 | 0.04 | 0.3 | 0.0 | 2 |
| UGB2-UGB3 26 | 0.0018 | 0.0000 | 1 | 1.26 | 0.25 | 0.0000 | 0 | 4.4 | 0.5 | 2 |

TABLE 2
Chemical-physical properties of the polymer hydrogel building
blocks (components);

| | | | | | Number |
|---|---|---|---|---|---|
| | | | | Number | of |
| | | | | of | Sulfate/ |
| | | | | Sulfate/ | Sulfonate |
| | | MWP | MWWE | Sulfonate | per |
| | | in | in | per | polmer |
| Polymer | Abbreviation | g/mole | g/mole | WE | molecule |
| Heparin (HEP) | GB1 | 14,000 | 540 | 2.7 | 70.2 |
| N-desulfated | GB2 | 13,600 | 523 | 1.8 | 48.4 |
| Heparin (N-DSH) | | | | | |
| 6O, N-desulfated | GB3 | 12,320 | 474 | 0.9 | 23.4 |
| Heparin (6ON- | | | | | |
| DSH) | | | | | |
| poly(4- | GB4 | 20,000 | 660 | 3.0 | 90.0 |
| styrenesulfonic | | | | | |
| acid-co-maleic | | | | | |
| acid), molar ratio | | | | | |
| sulfonic acid to | | | | | |
| maleic acid: 3:1 | | | | | |
| poly (4- | GB5 | 20,000 | 265 | 1.0 | 75.0 |
| styrenesulfonic | | | | | |
| acid-co-maleic | | | | | |
| acid), molar ratio | | | | | |
| sulfonic acid to | | | | | |
| maleic acid: 1:1 | | | | | |
| 4-Arm-PEG, | UGB1 | 10,000 | 44 | 0.0 | 0.0 |
| amin-terminated | | | | | |
| 4-Arm-PEG, | UGB2 | 10,000 | 44 | 0.0 | 0.0 |
| thiol-terminated | | | | | |
| 4-Arm-PEG, | UGB3 | 10,000 | 44 | 0.0 | 0.0 |
| maleimid- | | | | | |
| terminated | | | | | |
| 4-Arm PEG, | UGB4 | 15,600 | 44 | 0.0 | 0.0 |
| terminated with | | | | | |
| an enzymatically- | | | | | |
| cleavable peptid | | | | | |
| sequence | | | | | |

MWP: molar mass of the polymer,
MWWE: molar mass of the repeat unit:

TABLE 3
Chemical-physical properties of the signaling molecules, binding or sequestration of the
proteins in the hydrogel of the types UGB1-GB1 01, UGB1-GB2 02, UGB1-GB5 23, UGB1-GB3 03,
UGB1-GB4 04, UGB2-GB4 20 and the uncharged hydrogel UGB2-UGB3 26 (as a negative control)

| | | | Physical-chemical | | | | | | | |
|---|---|---|---|---|---|---|---|---|---|---|
| | | | properties | | | Structural | Binding of proteins in % | | | |
| | | | | | Number of | properties | of hydrogel with name | | | |
| | | | Molekular | | positiv | PROSITE- | UGB1-GB1 01 | | UGB1-GB2 02 | |
| | | Uniprot | weight in | | charged | Sequence | Average value | | Average value | |
| | Name | ID | Dalton | IEP | amino- | Families | SD | | SD | |
| Growth | FGF-2 | P09038 | 16407.6 | 9.6 | 28 | PS00247 | 31.0% | 3.2% | 5.4% | 8.7% |
| factors | TGFb1 | P01137 | 12794.7 | 8.6 | 15 | PS00250 | 18.0% | 4.2% | 0.0% | 2.0% |
| | bNGF | P01138 | 13434.2 | 9.0 | 21 | PS00248 | 83.5% | 7.3% | 78.5% | 4.5% |
| | EGO | P01133 | 6222.0 | 4.8 | 7 | PS50028 | 7.2% | 6.1% | 8.1% | 3.0% |
| | HGF | P14210 | 53683.7 | 7.7 | 78 | PS50070 | 41.7% | 4.0% | 48.3% | 5.2% |
| | PDGF-BB | P01127 | 12294.4 | 9.4 | 19 | PS50275 | 100.8% | 10.1% | 100.0% | 6.5% |
| | PLGF | P48763 | 22785.9 | 8.4 | 31 | PS50279 | 2.3% | 3.3% | 6.9% | 6.3% |
| | VEGF-A | P49763 | 23894.4 | 9.2 | 47 | PS50280 | 90.0% | 1.1% | 85.6% | 1.1% |
| Chemokines | Eotaxin | P15692 | 8384.8 | 9.9 | 13 | PS00472 | 100.0% | 0.0% | 98.0% | 0.4% |
| | GRO-alpha | P51671 | 7805.1 | 9.6 | 12 | PS00471 | 98.4% | 0.5% | 79.3% | 0.9% |
| | IL-5 | P09341 | 8922.3 | 8.2 | 17 | P800471 | 94.2% | 1.3% | 69.6% | 2.1% |
| | IP-10 | P02778 | 8646.2 | 10.2 | 17 | PS00471 | 98.2% | 0.5% | 92.7% | 2.3% |
| | MCP-1 | P13500 | 8684.9 | 9.4 | 14 | P800472 | 94.6% | 1.0% | 88.4% | 0.7% |
| | MIP-1alpha | P10147 | 7718.5 | 4.8 | 8 | PS00472 | 58.2% | 2.7% | 41.1% | 1.2% |
| | MIP-1beta | P13235 | 7818.7 | 4.8 | 8 | P500472 | 56.1% | 2.2% | 38.0% | 2.7% |
| | RANTES | P13501 | 7503.8 | 9.3 | 11 | P500472 | 99.0% | 0.2% | 95.2% | 0.6% |
| | SDF-1 alpha | P48061 | 8525.1 | 10.1 | 17 | PS00471 | 98.6% | 0.3% | 90.5% | 0.6% |
| Cytokines | GM-CSF | P04141 | 14477.4 | 5.2 | 15 | PS00702 | 8.4% | 2.1% | 3.9% | 10% |
| | IFN-gamma | P01579 | 16177.3 | 9.5 | 28 | | 99.5% | 0.1% | 92.6% | 2.0% |
| | IL-1beta | P01584 | 17376.7 | 5.9 | 19 | PS00253 | 27.0% | 3.7% | 25.2% | 0.6% |
| | IL-10 | P22301 | 18347.2 | 7.8 | 25 | PS00520 | 32.7% | 9.7% | 14.7% | 6.1% |
| | IL-12p40 | P29460 | 3469.6 | 5.4 | 43 | | 59.9% | 5.1% | 65.1% | 1.8% |
| | IL-4 | P05112 | 14963.0 | 9.3 | 28 | PS00838 | 98.5% | 0.3% | 82.41% | 2.0% |
| | IL-5 | P03231 | 20812.5 | 6.2 | 25 | PS00254 | 24.3% | 4.8% | 7.0% | 4.9% |
| | TNF-alpha | P01375 | 17362.8 | 7.0 | 18 | PS00251 | 38.9% | 3.5% | 24.7% | 3.3% |
| | Sclerostin | O9BOB4 | 21522.1 | 9.6 | 38 | | 65.0% | 3.1% | 42.0% | 11.2% |
| | DKK1 | O94907 | 25777.6 | 8.7 | 42 | | 84.0% | 6.2% | 78.0% | 3.2% |
| | Classified as TYPE according to Table 5.1 | | | | | | TYPE 1 | | TYPE 2 | |
| | Binding of proteins in % of hydrogel with name | | | | | | | | | |
| | UGB2-GB5 23 | | UGB1-GB3 03 | | UGB1-GB4 04 | | UGB2-GB4 20 | | UGB2-GB3 26 | |
| | Average value | | Average value | | Average value | | Average value | | Average value | |
| | SD | | SD | | SD | | SD | | SD | |
| Growth | | | 0.0% | 19.7% | | | | | | |
| factors | | | 0.0% | 5.9% | | | | | | |
| | 86.8% | 0.9% | 71.4% | 3.8% | 97.3% | 2.2% | 100.0% | 0.0% | | |
| | 11.6% | 9.9% | 11.5% | 0.9% | 28.3% | 3.0% | 36.6% | 3.4% | | |
| | 49.6% | 14.6% | 20.5% | 1.5% | 41.3% | 11.8% | 60.2% | 4.0% | | |
| | 82.9% | 8.5% | 100.0% | 2.2% | 100.0% | 5.5% | 100.0% | 2.0% | 19.7% | 23.1% |
| | | | 7.7% | 1.1% | 25.0% | 1.7% | | | | |
| | 98.3% | 2.3% | 69.4% | 4.5% | 93.4% | 1.3% | 93.7% | 2.9% | 1.9% | 4.7% |
| Chemokines | 98.4% | 1.0% | 91.9% | 2.3% | 100.0% | 0.0% | 99.2% | 9.2% | <0.1% | 5.1% |
| | 96.8% | 2.1% | 57.9% | 2.7% | 100.0% | 0.0% | 97.3% | 1.7% | <0.1% | 8.5% |
| | 94.5% | 7.3% | 44.7% | 2.5% | 99.0% | 0.6% | 97.6% | 1.7% | <0.1% | 1.0% |
| | 94.7% | 4.1% | 85.6% | 7.7% | 98.0% | 0.6% | 93.7% | 1.5% | <0.1% | 16.4% |
| | 99.8% | 1.8% | 45.3% | 4.3% | 98.7% | 0.7% | 97.4% | 1.4% | <0.1% | 8.3% |
| | 81.8% | 12.1% | 28.7% | 3.7% | 97.8% | 0.9% | 94.8% | 2.0% | <0.1% | 1.7% |
| | 78.0% | 15.7% | 23.3% | 3.1% | 98.4% | 0.7% | 94.0% | 4.1% | 0.2% | 1.0% |
| | 94.7% | 2.4% | 90.2% | 0.5% | 99.5% | 0.2% | 97.0% | 1.3% | <0.1% | 1.5% |
| | 99.9% | 1.9% | 80.6% | 1.5% | 99.7% | 0.3% | 97.2% | 1.6% | <0.1% | 2.9% |
| Cytokines | 7.5% | 14.1% | 11.1% | 2.5% | 24.2% | 1.7% | 36.0% | 4.5% | 1.3% | 4.2% |
| | | | 75.8% | 14.8% | 99.0% | 0.9% | | | | |
| | 5.5% | 8.1% | 25.2% | 3.6% | 72.0% | 1.9% | 59.8% | 5.0% | <0.1% | 5.4% |
| | 15.7% | 11.8% | 15.5% | 5.0% | 98.8% | 0.9% | 95.4% | 4.5% | <0.1% | 2.0% |
| | | | 45.8% | 1.3% | 92.5% | 1.3% | | | | |
| | 95.9% | 7.3% | 72.6% | 0.7% | 99.1% | 0.5% | 96.8% | 2.6% | <0.1% | 2.0% |
| | 49.4% | 26.7% | 17.3% | 7.8% | 89.5% | 0.5% | 88.2% | 4.6% | <0.1% | 1.0% |
| | | | 24.8% | 3.7% | 97.6% | 1.1% | | | | |
| | | | 0.0% | 8.6% | | | | | | |
| | | | 36.0% | 10.1% | | | | | | |

| | | | | |
|---|---|---|---|---|
| TYPE 2 | TYPE 3 | TYPE 4 | TYPE 4 | TYPE 5 |

TABLE 4
Activation time of the carboxyl groups of GB 1 to 5 in min, gel
volume used for the binding studies

| | | Gel volume for |
|---|---|---|
| | activation time | binding studies |
| UGB1-GB1 01 | 15 minutes | 12.14 μl |
| UGB1-GB2 02 | 10 min | 12.38 μl |
| UGB1-GB3 03 | 45 min | 8.56 μl |
| UGB1-GB 5-19 | 1 min | 12.00 μl |
| UGB2-GB4 20 to | 0 min | 10.00 μl |
| UGB2-UGB3 26 | | |

TABLE 5.1
Gel types with parameter ranges

| | A | B | C | D |
|---|---|---|---|---|
| Type 1 | 0.0040-0.0060 | 0.09-0.20 | <20 | uncharged |
| Type 2 | 0.0025-0.0040 | 0.05-0.18 | <20 | uncharged |
| Type 3 | 0.0005-0.0025 | 0.01-0.12 | <20 | uncharged |
| Type 4 | 0.0040-0.0100 | 0.16-0.80 | <20 | uncharged |
| Type 5 | 0 | 0 | <20 | uncharged |

TABLE 5.2
Gel types of preferred embodiments

| | A | B | C | D |
|---|---|---|---|---|
| Type 1 (UGB1-GB1 01) | 0.0050 | 0.12 | <20 | uncharged |
| Type 2 (UGB1-GB2 02) | 0.0035 | 0.12 | <20 | uncharged |
| Type 2 (UGB2-GB5 23) | 0.0038 | 0.14 | <20 | uncharged |
| Type 3 (UGB1-GB3 03) | 0.0019 | 0.06 | <20 | uncharged |
| Type 4 (UGB1-GB4 04) | 0.0045 | 0.28 | <20 | uncharged |
| Type 4 (UGB2-GB4 20) | 0.0045 | 0.16 | <20 | uncharged |
| Type 5 (UGB2-UGB3 26) | 0 | 0 | <20 | uncharged |

TABLE 6.1

| | e | F | G | C | D |
|---|---|---|---|---|---|
| Type 1 | 60-80 | 0.0015-0.0025 | 0.09-0.20 | <20 | uncharged |
| Type 2 | 30-75 | .0015-.0030 | 0.05-0.18 | <20 | uncharged |
| Type 3 | 10-30 | 0.0010-0.0040 | 0.01-0.12 | <20 | uncharged |
| Type 4 | 80-120 | 0.0018-0.0050 | 0.16-0.80 | <20 | uncharged |
| Type 5 | 0 | 0 | 0 | <20 | uncharged |

TABLE 6.2

| | e | F | G | C | D |
|---|---|---|---|---|---|
| Type 1 (UGB1-GB1 01) | 70.2 | 0.0018 | 0.12 | <20 | uncharged |
| Type 2 (UGB1-GB2 02) | 48.4 | 0.0025 | 0.12 | <20 | uncharged |
| Type 2 (UGB2-GB5 23) | 75.0 | 0.0018 | 0.14 | <20 | uncharged |
| Type 3 (UGB1-GB3 03) | 23.4 | 0.0027 | 0.06 | <20 | uncharged |
| Type 4 (UGB1-GB4 04) | 90.0 | 0.0031 | 0.28 | <20 | uncharged |
| Type 4 (UGB2-GB4 20) | 90.0 | 0.0018 | 0.16 | <20 | uncharged |
| Type 5 (UGB2-UGB3 26) | 0 | 0 | 0 | <20 | uncharged |

In Tables 5.1, 5.2, 6.1 and 6.2, the number of sulfate or sulfonate groups per repeat unit (WE) divided by the molar mass (MW) of the WE is indicated in column A as a property of the charged units in [mole/g]. As properties of the swollen hydrogels, the concentration of the sulfate or sulfonate groups in mmole/ml is given in column B and the storage modulus in kPa is given in column C. Column D lists the properties of the uncharged building blocks.

Column E indicates the molar concentration of the sulfate or sulfonate groups per mole of polymer (in mole/mole) of the charged building block. Column F shows the properties of the hydrogels as concentration of GB in mmole/ml, column G shows the concentration of sulfate or sulfonate groups in mmole/ml and column C as storage module.

The storage modulus of 20 kPa corresponds, according to the assumptions listed in the methods, to a mesh size of approximately 6 nm and thus should allow steric accessibility of all the structurally similar signaling molecules discussed herein into the hydrogels.

TABLE 7

| Name | Concentration/pg/ml |
|---|---|
| FGF-2 | 550 |
| TGFb1 | 4047 |
| bNGF | 29500 |
| EGF | 11986 |
| HGF | 30600 |
| PDGF-BB | 26000 |
| PIGF | 9971 |
| VEGF-A | 48290 |
| eotaxin | 2500 |
| GRO-alpha | 9500 |
| IL-8 | 12386 |
| IP-10 | 8800 |
| MCP-1 | 6500 |
| MIP-1 alpha | 16400 |
| MIP-1 beta | 13500 |
| RANTES | 3600 |
| SDF-1 alpha | 39800 |
| GM-CSF | 41000 |
| IFN-gamma | 50700 |
| IL-1 beta | 8250 |
| IL-10 | 9250 |
| IL-12p40 | 13500 |
| IL-4 | 36200 |
| IL-6 | 44368 |
| TNF-alpha | 31363 |
| sclerostin | 13300 |
| DKK1 | 864 |

Table 7 lists substances of substance groups A and B in relevant concentrations in the biofluids.

The invention will now be explained with reference to the exemplary embodiments (AB) in the tables shown above.

For example, the hydrogels from UG B1-GB1 01 in Table 1 (AB1) have proven to be particularly advantageous for adjusting the concentrations of different signaling molecules (see Table 3).

The class of chemokines has a high structural similarity to the tertiary structure, which is stabilized by the interaction of four cysteines by disulfide bridges (corresponding to PROSITE ID: PS00471 and PS00472). Furthermore, chemokines are characterized by a strong net positive charge IEP>9 or positively charged domains such as MIP1-alpha and MIP1-beta and a molar mass less than 10 kDa (see Table 3) and are strongly bound in the hydrogel by this hydrogel type and thus depleted from adjacent biofluids (binding of MIP1-alpha and MIP1-beta approximately 60%, the other chemokines eotaxin, GRO-alpha, IL-8, IP-10, MCP-1, RANTES and SDF-1 alpha are ≥95% bound (Table 3), while surprisingly the following highly positively charged signaling molecules FGF-2 (IEP 9.58) and TGFb1 (IEP 8.59) associated with the class of growth factors and structurally similar proteins of the FGF family (corresponding to PROSITE ID: PS00247) and of the TGFb1 family (corresponding to PROSITE ID: PS00250) are bound only very weakly with 31% and 18%, PLGF (IEP 8.37) with a binding <10% even less (Table 3), and the signaling molecules associated with the class of cytokines with a slightly basic IEP, such as IL-10 (IEP 7.65), as well as signaling molecules with an IEP in the neutral range (pH 5.5-7), such as IL1-beta, IL6 and TNF-alpha and structurally similar proteins of the IL-1-beta protein in family (corresponding to PROSITE ID: PS00253), the IL-10 protein family (corresponding to PROSITE ID: PS00520), the interleukin-6/GM-CSF/MGF family (corresponding to PROSITE ID: PS00254) and TNF-alpha protein family (corresponding to PROSITE ID: PS00251) are bonded only weakly ≤30% (Table 3) from the complex biofluid (binding of TNF-alpha ca. 38%) and are thus present in the biofluid in almost unchanged concentrations. Furthermore, the growth factor with a slightly negative net charge EGF (IEP 4.8) and binding of less than 10% is almost not bonded. As a result, signaling factors essential for the survival of cells, such as EGF, FGF-2, TGFβ or PLGF, remain unaffected by the sequestration realized by way of the abovementioned hydrogels.

The strongly basic cytokines IFN-gamma (IEP 9,52), IL-4 (IEP 9,25), the WNT and BMP antagonists sclerostin (9,57) and DKK1 (8,72) as well as structurally similar proteins of IL-4/IL-13 family (corresponding to PROSITE ID: PS00838) and the sclerostin-like family (corresponding to InterPro ID: IPR008835) and the strongly basic growth factors bNGF (IEP 9.00), PDGF (IEP 9.4) and VEGF (IEP 9,2) and structurally similar proteins of the NGF family (corresponding to PROSITE ID: PS00248) and of the PDGF family (corresponding to PROSITE ID: PS00250) and IL-12p40 are strongly bound at ≥59.9% as expected (Table 3) and therefore depleted from the biofluid.

By contacting a biofluid with a hydrogel with the specified charge and network characteristic of the type 1 according to Table 5.1, a selective selection of growth factors having proteins structurally similar to DKK1, bNGF, PDGF-BB, VEGF, as well as chemokines (e.g. eotaxin, Gro-alpha, IL-8, IP-10, MCP-1 alpha, MIP1 beta, Rantes, SDF1 alpha) and cytokines with proteins structurally similar to IFN-gamma, IL4 and sclerostin according to annotated sequence motifs and protein families can be depleted from the biofluid or their concentration in the biofluid can be increased by precharging the hydrogel, while advantageously leaving nearly unchanged the concentration of the above-mentioned signaling molecules EGF, FGF-2, TGFb1, PLGF, IL-10, IL1 beta, IL6, and TNF alpha. In addition, this hydrogel has an interaction of the GB1 with the enzymes thrombin and antithrombin important for blood clotting according to Uwe Freudenberg et al., Journal of Controlled Release, Journal of Controlled Release: Official Journal of the Controlled Release Society 220, no. Part A (Dec. 28, 2015): 79-88, doi: 10.1016/j.jconrel.2015.10.028.

Another particularly advantageous exemplary embodiment (AB2) consists in the hydrogel type UGB1-GB2 02 (see Table 2) which has a binding or sequestering pattern that is nearly identical to the hydrogel type UGB1:GB1 01 (see Table 3), except for the weaker binding of the two chemokines MIP-1 alpha and MIP-1 beta and sclerostin (each about 40%, Table 3) and the even weaker binding of IL-10 (15%), IL-6 (8%), TNF alpha (25%), see Table 3, and the more advantageous almost non-existent binding of FGF-2 (5%) and TGFb1 (0%), and the lack of interaction of GB2 with the blood clotting enzymes thrombin and antithrombin (see Uwe Freudenberg et al., Journal of Controlled Release, Journal of Controlled Release: Official Journal of the Controlled Release Society 220, no. Part A (Dec. 28, 2015): 79-88, doi: 10.1016/j.jconrel.2015.10.028). Another advantageous exemplary embodiment is UGB2-GB5 23 (AB 23, see Table 1), which has a charge characteristic that can like the AB2 also be assigned to type 2 in accordance with Table 5.1, but which has a slightly different sulfonate content of 0.14 mmole/ml (Table 1 or Table 5.2, column B) and a slightly different number of sulfonate groups per repeat unit (WE) divided by the molar weight (MW) of the WE in [mole/g] of 0.0038, however with both parameters the range given in Table 5.1 for a type 2 hydrogel. The hydrogel, however, was formed from another charged, synthetic gel building block (GB5) with a different crosslinking reaction (crosslinking type 2, Table 1) and has a nearly identical binding or sequestering patterns as the AB2 (see Table 3) except for the somewhat stronger binding of the two chemokines MIP-1 alpha and MIP-1 beta. This confirms the classification of the hydrogels with the characteristic charge patterns according to Table 5.1 and also represents a particularly advantageous sequestration pattern.

Another advantageous exemplary embodiment (AB3) is represented by the hydrogel type UGB1-GB3 03 (see Table 2), which has a significantly weaker binding to many proteins (signaling molecules) due to the significantly lower concentration of sulfate or sulfonate groups in the hydrogel (Table 5.2, column B, 0.06 compared to 0.12 mmole/ml for AB1 or AB2) and also the lower number of sulfate or sulfone groups per WE divided by the molar mass of the WE of 0.0019 compared to 0.005 or 0.0035 for AB 1 or AB2 (see Table 5.2, Column A) compared to the AB1 and AB2. For this type of hydrogel, bNGF, PDGF-BB, and VEGF A, eotaxin, GRO-alpha, IP-10, Rantes, SDF-1 alpha, IFN-gamma, and IL-4 are more than 50% bound by the hydrogel and therefore depleted from a biofluid. In contrast, IL-8, MCP-1 and IL-12p40 are only bound by the hydrogel by about 45% (see Table 3), HGF, MIP-1 alpha, MIP-1 beta, IL-1 beta, IL-10, IL-6, TNF alpha and DKK1 by 16-36% only weakly (see Table 3), and FGF-2, TGFb1, EGF, PLGF, GM-CSF and sclerostin almost not (<12%) (see Table 3) and the majority of signaling molecules is therefore significantly less than for AB1 and AB2. With this type of gel, for example, a largely selective separation of bNGF, PDGF-BB, and VEGF A, eotaxin, GRO-alpha, IP-10, Rantes, SDF-1 alpha, IFN-gamma and IL-4 from arbitrary biofluids is possible.

Another advantageous embodiment is the hydrogel UGB1-GB4 04 (see Table 1), which due to the high concentration of sulfate or sulfonate groups of 0.28 mmole/ml in the hydrogel (Table 5.2, column B) and a likewise high number of sulfonate groups per WE divided by the molar mass of WE of 0.0045 mole/g, with the exception of HGF, PLGF, GM-GSF and EGF, binds all investigated factors with a high efficiency (between 72-100%, see Table 3) in the hydrogel and thus depletes them from the biofluid (see Table 3).

In particular, with this hydrogel, a depletion of all investigated and structurally similar factors (IEP and the presence of structural similarities) from adjacent biofluids occurs, particularly the class of chemokines, which have high structural similarity of the tertiary structure resulting from the interaction of four cysteines by disulfide bridges (corresponding to PROSITE ID: PS00471 and PS00472) and which are characterized by a strongly positive net charge IEP>9 or positively charged domains as in MIP1 alpha and MIP1 beta and a molecular weight of less than 10 kDa (see Table Protein properties), FGF-2 (IEP 9.58), TGFb1 (IEP 8.59) and structurally similar FGF family proteins (corresponding to PROSITE ID: PS00247), the TGF family (corresponding to PROSITE ID: PS00250) and the class of signaling molecules associated with cytokines having slightly basic IEP such as IL-10 (IEP 7.65) as well as signaling molecules with an IEP in the neutral range (pH 5.5-7), such as IL1β, IL6, and TNF and structurally similar proteins of the IL-1β protein family (corresponding to PROSITE ID: PS00253) and of the IL-10 protein family (corresponding to PROSITE ID: PS00520). Another advantageous exemplary embodiment, UGB2-GB4 20 (see Table 1), which with a charge characteristics that can according to Table 5.1 also be associated with type 4 like AB4, which has the same GB 5, see Table 5.1 column A: 0.0045, but a lower sulfonate concentration of 0.16 mmole/ml (see Table 1 and Table 5.1) that is still attributable to the type 4, but was formed with a different crosslinking reaction (VN: 2 in Table 1), has a nearly identical binding or sequestration patterns to the UGB1-GB4 04 (see Table 3). This result thus also confirms the effective predictive power of the charge characteristics of types 1-5 according to Table 5.1 for the differentiated sequestration of substances or groups of substances.

Another exemplary embodiment is the uncharged hydrogel (UGB2-UGB326) formed from UGB1 and UGB3 which, as expected, binds as a negative control almost none of the signaling molecules from the biofluid. Except for the minor sequestration of PDGF-BB of 19.7±23.1% and IP 10 of 16.1±16.4%, which cannot be classified as significant due to the large standard deviations, no sequestration (≤1.3% for all signaling molecules tested, see Table 3) occurs for this hydrogel. This result can be clearly attributed to the absence of charged affinity centers and thus to the absence of the afore-discussed charge interactions. The storage modulus and hence also the network mesh size, however, of 4.4 kPa (Table 1) are almost identical to the storage moduli of UGB1-GB2 02, UGB2-GB4 20 and UGB2-GB5 23, i.e. the steric interactions with the charged exemplary embodiments the signaling molecules are comparable. These results, in particular by the lack of interactions (i.e. the absence of binding and sequestering effects) of the uncharged hydrogel (UGB2-UGB3 26) with the signaling molecules, substantiate the claims for differentiated sequestration according to Table 5.1.

At the same time, individual factors can be released into the biofluid via by selectively precharging the hydrogels of the types 1 to 3 of Table 5 in parallel and independently of sequestering and hence depletion of the other factors. Thus, with the various types of hydrogels, almost any level of individual signaling molecules in complex biofluids can be adjusted by targeted precharging of the hydrogels or the nearly quantitative sequestration (depletion) of signaling molecules (e.g. application for separating signaling molecules from any protein-containing solution).

Based on the exemplary embodiments in UGB1-GB1 01 to UGB2-UGB3 26, the following relationships between protein properties, hydrogel properties and binding of proteins in the hydrogel network important for the description of the invention are evident: A strong binding of the signaling molecules according to Table 3 correlates on the side of proteins with a net positive charge (and here with a high, basic IEP as the corresponding parameter) as well as with the inverse of the molecular weight (high IEP and small molecular weight enhance binding, see Table 3, the highly positively charged and relatively small chemokines (<9 kDa) are most strongly bound and on the side of the hydrogel network, (1) a high concentration of sulfate or sulfonate groups in the hydrogel (Table 1 and 5.2, column B), and (2) a high number of sulfate or sulfonate groups per WE divided by the molar mass of the WE enhance the binding of the signaling molecules. Commensurate with these parameters, the following sequential order results for the hydrogels from the strongest binding to the weakest binding: UGB1-GB4 04≈UGB2-GB4 20<UGB1-GB1 01<UGB1-GB2 02≈UGB2-GB5 23<UGB1-GB3 03<<UGB2-UGB3 26. This result is supported by the experimentally found binding values (see Table 3). In addition, with different combinations of the aforementioned hydrogel network parameters (1) and (2), the strong interactions known in the literature as “specific interactions”—i.e. a spatial matching of the different interaction centers, between proteins and negatively charged polyelectrolytes (e.g. glycose-aminoglycans) can be adjusted, and other structural properties in addition to the molecular weight and net charge can thus also be used on the protein side for modulating the binding to the hydrogels. These correlations explain also the low binding of the strongly basic and relatively small signaling molecules FGF-2, TGFb1 and PLGF to the hydrogel types UGB1-GB1 01, UGB1 GB2-02, GB3 UGB1-03 and UGB1-GB4 04.

Further possibilities for controlling the levels of soluble signaling molecules are based on the sequential or combined use of the different hydrogel types in order to precisely adjust a wide variety of specific sequestering and release properties. By targeted variation of the network properties (in particular the previously described parameters (1) and (2) and also the combination of hydrogel micro-particles of different composition (e.g. charging with individual signaling molecules) in so-called multiphase hydrogel materials (i.e. by mixing hydrogel types of different types or precharging with signaling molecules or proteins), other advantageous concentration ratios can also be set in adjacent biofluids.

By extending the analysis to further signaling molecules, the proposed method also allows the control of the concentration of these substances in any type of biofluids.

In order to be able to effectively control the complex biological functions of signaling molecules, for example for therapeutic concepts, an active management of different signaling molecules from the immediate biological environment of the cells or the biological application is necessary in addition to the sustained release of individual signaling molecules from the hydrogel precharged with the signaling molecules. The interactions with a large number of relevant signaling molecules must therefore be considered. When hydrogels containing sulfated or sulfonated components are charged, for example, with a signaling molecule for sustained release, other therapeutically important molecules from the biological environment can be simultaneously sequestered and thus inactivated simultaneously with the therapeutically desired release of the signaling molecules, thus representing an undesirable side effect. On the other hand, however, it is also possible to specifically sequester and to thus inactivate therapeutically undesired signaling molecules into hydrogel carrying the sulfated or sulfonated groups. Overall, very complex release and sequestration scenarios are possible, which decide in their totality about the biological effect, in particular the molecular separation properties of the materials.

Further advantageous embodiments of the invention include the use of hydrogels according to the types 1-4 of Table 5 for modulating the concentration or levels of biologically active proteins at low total levels.

An essential advantage of the method lies in the universality of its application possibilities. The method can advantageously be used both for biotechnological purification and for separation of protein mixtures from or in biofluids by using hydrogels with sulfated or sulfonated components.

In an exemplary narrower sense, the method for factor management can be used in vivo for controlling the angiogenesis, immune diseases, diabetes, neurodegenerative diseases and wound healing.

In wound healing, the mostly pro-inflammatory acting chemokines (e.g. eotaxin, GRO-a, IL-8, IP-10, MCP-1, MCP-3, MCD, Rantes and SDF-1) are sequestered inside the hydrogels, whereas pro-regenerative factors (e.g. EGF, FGF-2, TGFb1, IL-10, HGF and PLGF) will remain largely unaffected.

A particularly important field of application of the invention is the control of the concentrations of substances in biofluids, which are responsible for deciding the fate of cells in vitro and in vivo. The dysregulation of pro-inflammatory-acting chemokines and the associated chronic inflammation is the cause of the development of various diseases such as Crohn's disease, ulcerative colitis, multiple sclerosis, asthma or rheumatoid arthritis. The targeted modulation of the concentration of these inflammatory factors from biofluids by the application of different hydrogels with sulfated or sulfonated components according to the invention represents a possible application scenario.

In a broader sense, the application of the hydrogels with sulfated or sulfonated components according to the invention allows a targeted purification of the here investigated or structurally similar signaling molecules from complex protein mixtures. The described gel systems can thus be biotechnologically used for the targeted purification of proteins from cell lysates of microbial or eukaryotic origin. In this case, these cell lysates are prompted by hydrogels according to the invention to bind the signaling molecules. In a second step, the bound substances can be removed again from the hydrogels for further use by rinsing with highly concentrated saline solutions or positively charged polyelectrolytes (e.g. chitosan) and thus separated. Furthermore, a negative selection for the separation of the signaling molecules, which are under these conditions weakly bound to the hydrogels of types 1-3 binding example. EGF, FGF-2, TGF-.beta., IL-10, HGF and PLGF, is possible by using supernatants after 24 h binding.

A relevant practical advantage of the invention is that the binding of a variety of biologically relevant signaling molecules to sulfated or sulfonated hydrogels with targeted graduated charge characteristic and network structure is realized even at high albumin concentrations of 1 mg/ml to 45 mg/ml at simultaneously low levels of signaling molecules from 100 pg/ml to 2000 ng/ml in a physiological electrolyte as a biofluid, and that as a result selectivity differences in the binding of the individual signaling molecules to the different hydrogel types can be employed.

The exemplary embodiments of UGB1-GB1 01 to UGB1-GB4 011 given in Table 1 are covalently crosslinked hydrogels according to crosslinking reaction 1, wherein 6 to 24 carboxyl groups (depending on the molar ratio of 1.5 to 6 in four-arm star PEG) of a molecule of GB4 (Poly (4-styrenesulfonic acid-co-malic acid), molar ratio of styrene sulfonic acid: maleic acid 3:1, Table 1) were activated with EDC/sulfo-NHS and reacted directly with the four-arm, amine-terminated UGB1 to yield a hydrogel (see Table 1), resulting in hydrogels with a variable sulfonate concentration of 0.28 to 0.06 mmole/ml and variable storage moduli of 4.8 to 19.6 kPA (see Table 1).

The exemplary embodiments given in Table 1 UGB1-GB5 12 to UGB1-GB5 19 are covalently crosslinked hydrogels according to crosslinking principle 1 in which 6 to 24 carboxyl groups (depending on the molar ratio 1.5 to 6 in four-functional star-PEG) of a molecule of poly (4-styrenesulfonic acid-co-maleic acid) with the molar ratio of styrenesulfonic acid to maleic acid 1:1 (GB5), were activated by EDC/SN HS and converted directly to a hydrogel by means of the four-arm, amine-terminated UGB1 (see Table 1), resulting in hydrogels with a variable sulfonate concentration of 0.13 to 0.05 mmole/ml and variable storage moduli from 3 to 21.8 kPA (see Table 1).

The exemplary embodiments UGB2-GB4 20 to UGB2-GB4 22 are covalently crosslinked hydrogels according to crosslinking principle 2, in which eight of the carboxyl groups of the GB4 (poly (4-styrenesulfonic acid c o-maleic acid) with the molar ratio of styrenesulfonic acid:maleic acid 3:1) and activated with EDC/sulfo-NHS are in a first step functionalized with the short bifunctional crosslinker molecule N-(2-aminoethyl) maleimide and thereafter purified and isolated. These derivatives of GB4 were then converted to a hydrogel by mixing in physiological saline (or serum) with the UGB-2 (thiol-terminated four-arm PEG), resulting in hydrogels with a gradation in the concentration of sulfonate groups from 0.16 to 0.05 mmole/ml and the storage moduli of 3.7-0.2 kPa.

The exemplary embodiments UGB2-GB5 23 to UGB2-GB5 25 are covalently crosslinked hydrogels according to crosslinking principle 2 (VN2, Table 1) in which eight of the carboxyl groups of GB5 (poly (4-styrenesulfonic acid-co-maleic acid) with the molar ratio styrenesulfonic acid:maleic acid 1:1) and activated with EDC/sulfo-NHS are functionalized in a first step by the short bifunctional crosslinker molecule N-(2-aminoethyl) maleimide and subsequently purified and isolated. These derivatives of GB5 were then converted to a hydrogel by mixing in physiological saline solution (or serum) with the UGB-2 (thiol-terminated four-arm PEG), resulting in hydrogels with a gradation in the concentration of sulfonate groups of 0.14 to 0.04 mmole/ml and storage moduli of 4.6-0.3 kPa.

The exemplary embodiment UGB2-UGB3 26 is an uncharged PEG hydrogel in which, according to the crosslinking principle 2, thiol-terminated four-arm PEG (UGB-2) was reacted with a maleimide-terminated four-arm PEG (UGB-3). This hydrogel served in the sequestering experiments as an uncharged negative control.

Surprisingly, full-synthetic hydrogels having a large range of advantageous properties and combinations of properties and without disadvantageous immunogenic reactions can be prepared based on the exemplary embodiments 04-5. Thus, a broad concentration of sulfonate groups in the swollen hydrogel of 0.04 to 0.28 m mole/ml and with an equally large variation of the storage modulus 0.2 to 21.8 kPa is largely adjustable independent of each other. For example, the hydrogels UGB2-GB4 21, UGB1-GB5 14, UGB1-GB5 15 and UGB1-GB4 08 have a constant concentration of sulfonate groups in the swollen hydrogel of 0.08 mmole/ml, but at the same time increasing storage moduli of 1.1, 5.5, 12.5 and 19.6 kPa, i.e. the sulfonate concentration and the stiffness of the hydrogels can be modulated independently of one another over a wide range. Also, with the selection of UGB1-GB5 19 having a sulfonate concentration of 0.05, UGB1-GB4 11 having a sulfonate concentration of 0.06, and UGB1-GB4 10 having a sulfonate concentration of 0.09 with a nearly constant storage modulus of 8.0-8.5 kPa, the sulfonate concentration can be varied independently of the stiffness of the hydrogels (see Table 1). In addition, with a further variation of the molar ratios UGB:GB in the ranges between 1.5 and 3 and between 3 and 6 for the VN1 and between 0.5 and 0.75 and 1 and between 1.5 and 1.5 and 2 for the VN2 and a further variation of the concentration of the GB and UGB, many other combinations of properties can be produced.

Surprisingly, by crosslinking of the poly (4-styrenesulfonic acid-co-maleic acid), starting with the EDC/sulfoNHS activation of one of the closely adjacent acid groups in the maleic acid, which are influenced in an unpredictable manner in their reactivity not only by the adjacent carboxyl group but especially by the sulfonate groups and the hydrophobic styrene units, hydrogel materials defined with uncharged building blocks could be synthesized with the aforementioned widely gradable properties. Here, a precise network formation (for VN1) or derivatization (for VN2) was achieved by using a surprisingly very short activation time of 1 min, i.e. the time during which the EDC/sulfoNHS was added to poly (4-styrenesulfonic acid-co-maleic acid), see Table 4, before thereafter an amino-group-bearing molecule was reacted by mixing with the then activated poly (4-styrenesulfonic acid-co-maleic acid) and a very intensive intermixing of the reactants.

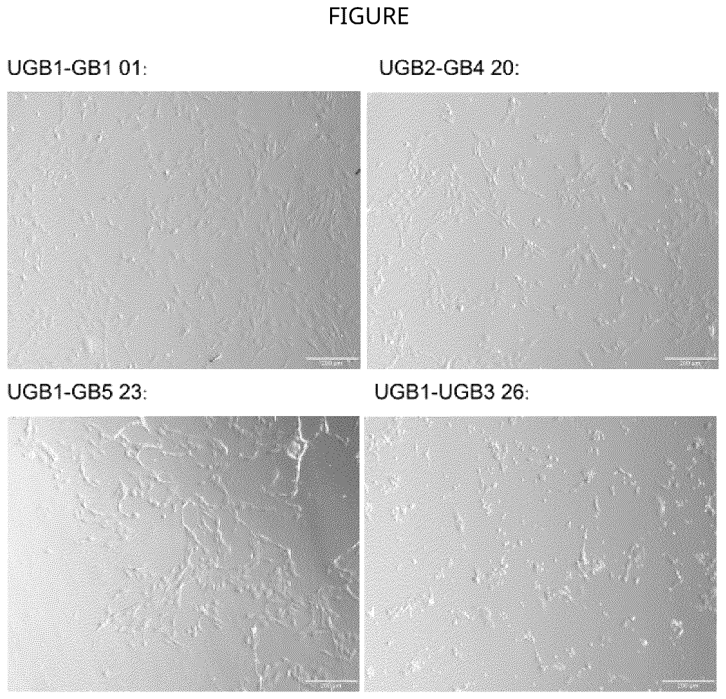

In another exemplary embodiment, hydrogels of the types UGB1-GB1 01, UGB1-GB4 20, UGB1-GB5 23 and UGB1-GB3 26 were functionalized with the adhesion peptide SEQ ID NO 22 and precharged with VEGF-A and FGF-2, and on used on the gel surface in a serum-free cell culture medium for the cultivation of human endothelia cells. The morphology of the adherent cells was analyzed after 24 h culture period, as shown in FIG. 1 for UGB1-GB1 01, UGB2-GB4 20, UGB2-GB5 23, and UGB2-UGB3 26. The endothelial cells assumed a different morphology on the different hydrogels, describable by the two parameters aspect ratio and circularity. A high aspect ratio and low circularity corresponds to the desired elongated endothelial cell morphology, the first step in the formation of the biologically desired tubular structures. The aspect ratio of the endothelial cells cultured on UGB1-GB1 01, UGB2-GB4 20 and UGB2-GB5 23 was 3.4±1.0, 4.1±1.1 and 4.9±1.6, while the uncharged reference gels UGB2-UGB3 26 had a significantly lower aspect ratio of 2.1±1.2. The circularity was correspondingly the inverse with 0.5±0.1, 0.4±0.1, 0.3±0.1 and 0.7±0.3 for endothelial cells on the hydrogels UGB1-GB1 01, UGB1-GB4 20 and UGB1-GB5 23 and UGB1-GB3 26. These differences were statistically highly significant. Accordingly, the endothelial cells according to Table 5.1 behave significantly different on the hydrogels with the different charge and sequestration properties of type 1 (UGB1-GB1 01), of type 4 (UGB2-GB4 20), of type 2 (UGB2-GB5 23) and of the uncharged type 5 (UGB1-GB3 26). On the uncharged hydrogel, the cells show undesirable short aspect ratios and high circularity, while the desired morphology increases in the order of UGB1-GB1 01<UGB1-GB4 20<UGB2-GB5 23. The hydrogel UGB2-GB5 23 of type 2 shows the best results in this experiment. This grading thus confirms a direct influence of the charge properties and sequestration patterns on the culture of human endothelial cells.

In another exemplary embodiment, hydrogels of type 1 (UGB1-GB1 01), of type 4 (UGB1-GB4 20), of type 2 (UGB1-GB5 23) and of the uncharged type 5 (UGB1-UGB3 26) were used for polymerization of human mesenchymal stromal cells. The hydrogels were cell-responsive, that is, in addition to the functionalization of the hydrogels with the adhesion-mediating peptide SEQ ID NO 21, the cleavable peptide sequence SEQ ID NO 23 enzymatically cleavable by matrix metalloproteases secreted by these cells was preconjugated on the respective uncharged building block and used for crosslinking according to VN2. The metabolic activity of the embedded cells was characterized after 24 hours by a PrestoBlue® test as evidence of viability. The metabolic activity, measured as relative fluorescence units, of the polymerized human mesenchymal stromal cells within the hydrogel of type 1 was 7112±3924, of type 2 3704±2945, of type 4 3160±6023 and of the uncharged type 5 2316±446, respectively. Accordingly, human mesenchymal stromal cells showed the highest metabolic activity when embedded in Type 2 gels and the lowest metabolic activity when embedded in gels of the uncharged reference gel. The hydrogels, especially of type 2, have thus proved to be particularly advantageous for the cultivation of living human mesenchymal stromal cells in 3 D.

In another advantageous exemplary embodiment, UGB1-GB1 01, UGB2-GB4 20, UGB2-GB5 23, and UGB2-UGB3 26 were functionalized by differentiated sequestration with the antibiotic gentamicin bearing positively charged groups. The inhibition was then measured by releasing the gentamicin from the different hydrogels using the two relevant pathogenic bacterial strains Escherichia coli (E. coli) and Staphylococcus epidermidis (Staph. Epidermidis). The antimicrobial activity was measured by the size of the inhibition zone (distance from the hydrogel on the culture plate, see Materials and Methods). No inhibition zones were detectable for the hydrogels of the types UGB1-GB101, UGB2-GB420, UGB2-GB523, and UGB2-UGB3 26 without prior gentamicin sequestration, i.e. the hydrogels did not have an antimicrobial effect. In the case of a prior differentiated sequestering of gentamicin by the hydrogels, the following graded inhibition zones were measured: UGB1-GB1 01: 3.08±0.33 mm, UGB2-GB4 20 E. coli: 3.08±0.33 mm, S epidermidis: 2.94±0.35 mm, UGB2-GB4 20: E. coli: 1.78±0.27 mm, S. epidermidis: 1.76±0.45 mm; UGB2-GB5 23 E. coli: 2.18±0.38 mm, S. epidermidis: 1, 81±0.51 mm, and for the uncharged reference gel UGB2 UGB3-26 E. coli: 0.48±0.36 mm, S. epidermidis: 0.90±0.28 mm. Accordingly, as expected, only very small amounts of gentamicin are sequestered nonspecifically for the uncharged reference gels UGB2-UGB3 26 and subsequently released again. For the other hydrogels, a graded antimicrobial action could be found in the order of UGB2-GB420 <UGB2-GB523 <UGB1-GB1 01. This result supports the claims for the differentiated sequestration of positively charged drug molecules. In addition, different antimicrobial effects can also be established by varying the sulfurization concentration of gentamicin.

### Material and Methods

### Region-Selective Desulfation of Heparin (Produced From GB2 and GB3 From GB1)

For the synthesis of region-selective desulfated of heparin (GB2 and GB3), heparin (MW 14,000, Merck Millipore, manufacturer no: 375095, Germany, GB1) was first dissolved in deionized, ultrapure water and desalinated with the aid of an Amberlite IR-120H+ ion exchange column (Sigma-Aldrich, Germany). By addition of pyridine (Sigma-Aldrich, Germany) to the heparin solution until pH 6 was reached, a heparin-pyridine salt was formed which, enriched by solvent evaporation in a B-490 rotary evaporator (Büchi, Germany), was subsequently lyophilized at −80° C. (GEA Lyovac GT2, Germany) and stored at −20° C. until further processing.

To prepare N-desulfated heparin (N-DSH, GB2), 1 g/L heparin-pyridine was dissolved in a mixture of DMSO and deionized ultrapure water (95:5) and incubated at 50° C. for 1.5 hours. The resulting solution was diluted 1:1 with deionized ultrapure water and adjusted to pH 9 with 1M sodium hydroxide solution (Sigma-Aldrich, Germany). After dialyzing the solution (Spectrum Labs, Germany, MWCO=8 kDa) against deionized, ultrapure water for three days, N-acetylation of desulfated heparin was performed. To this end, 10% v/v methanol (Sigma-Aldrich, Germany) and 50 mM sodium carbonate (Sigma-Aldrich, Germany) were added to the solution. The acetylation reaction was carried out for three hours with cooling to 4° C. and constant stirring at 400 rpm by addition of 400 μl acetic anhydride (Sigma-Aldrich, Germany) per 1 mg heparin every half hour and subsequent adjustment of the pH to 7.5 by addition of a 2 M sodium carbonate solution.

To prepare 6O-N-desulfated heparin (6ON-DSH, GB 3), 1 g/L heparin-pyridine was dissolved in a mixture of DMSO and deionized ultrapure water (95:5) and incubated at 90° C. for 24 hours. The resulting solution was diluted 1:1 with deionized, ultrapure water and adjusted to pH 9 with 1 M sodium hydroxide solution (Sigma-Aldrich, Germany). After dialyzing in a dialysis tubing (Spectrum Labs, Germany, MWCO=8 kDa) against deionized ultrapure water for three days, the 60-N-desulfated heparin was enriched by solvent evaporation B-490 (Büchi, Germany) and thereafter lyophilized (GEA Lyovac GT2, Germany).

### Characterization of GB and UGB (see Table 2):

The molecular weight of the GB1-GB3 was determined by multi-angle light scattering at 690 nm on a Dawn HELEOS II (Wyatt Technology Europe, Germany). The values dn/dc required for the molecular weight determination were determined with an RI detector (Optilab T-rEX, Wyatt, Germany) at a wavelength of λ=690 nm. The RI increments, dn/dc, of 0.1351 mL·g⁻¹ for GB1, GB2, and GB3 were needed for the evaluation of the light scattering experiments. The evaluation of the light scattering results for determining the molar mass was carried out with the Astra software, version 6.1 (Wyatt Technology, USA).

The degree of sulfation of the heparin derivatives (GB1-GB3) was determined by elemental analysis (Elementar, Vario MICRO cube, Germany) on the basis of the molar S:N ratio. Because each disaccharide unit contains exactly one N atom, the sulfation degree of GB1, GB2 and GB3 (number of sulfate groups/repeat unit) was determined by the molar ratio of S:N. Furthermore, the molar mass of the repeat unit was determined therefrom. The number of sulfate groups per repeat unit or per mole of polymer was calculated on this basis.

The molecular weight and the number of sulfonate groups per repeat unit or per mole of polymer of GB4 and GB5 (poly (4-styrenesulfonic acid-co-maleic acid)) are based on information from the manufacturer (Sigma-Aldrich, manufacturer no.: 434566, Germany).

The molecular weight and structure of UGB1, UGB2, UGB3, amino-, thiol- or maleimide-terminated four-arm PEG are based on information from the manufacturer (Jenkem Technology, USA). The molar mass and structure of UGB4 (enzymatically cleavable peptide-terminated four armed PEG) has been manufactured and used according to the process described by Tsurkan et al. 2013 (Adv. Mater. 2013, 25, 2606-2610).

### Preparation of the Hydrogels Based on Heparin or Heparin Derivatives (See Also Table 1):

To prepare the hydrogels, heparin (GB1) (Merck-Millipore, manufacturer no.: 375095, Germany), desulfated heparin derivatives based on GB1 were dissolved by previously described methods according to the hydrogel type (see Table 2) as the charged component (GB), see region-selective desulfation, for GB2 and GB3) in deionized, ultrapure water at 4° C. for 30 seconds at 500 rpm with a vortex mixer (IKA, Germany). In addition, four-arm, amine-terminated PEG (MW 10,000, Jenkem Technology, USA), 1-ethyl-3-(3-dimethylaminopropyl) carbodiimide (EDC; Sigma-Aldrich, Germany) and N-hydroxysulfosuccinimide (sulpho-NHS, Sigma-Aldrich, Germany) were dissolved in the same way for all types of hydrogels as UGB1. In order to ensure the complete solution of the hydrogel building blocks, these solutions were additionally treated for 5 min at 4° C. with an ultrasonic bath (RK 100H, Bandelin, Germany).

To activate the GB1 to GB 3, EDC and sulpho-NHS (2:1 ratio of EDC:sulpho-NHS) were added to the dissolved GB (4 mole sulpho-NHS, 8 mole EDC per mole of UGB1 of the final reaction mixture) and incubated at 4° C. according to the times listed in Table 4. After the activation time of GB1, GB2, GB3, the dissolved amino-terminated 4-arm PEG (UGB1) was additionally added and mixed for 30 seconds at 500 rpm with a vortex mixer (IKA, Germany).

Preparation of the Hydrogels Based on Poly (4-styrenesulfonic Acid-co-maleic Acid) After Crosslinking Reaction 1 (VN1, See Also Table 1):

Poly (4-styrenesulfonic acid-co-maleic acid) with the molar ratio of 4-styrenesulfonic acid: maleic acid of 3:1 (Sigma-Aldrich, manufacturer no.: 434566, Germany, GB4, see Table 2) and with the molar ratio of 4-styrenesulfonic acid:maleic acid of 1:1, Sigma-Aldrich, manufacturer no: 434558, Germany, GB5, see Table 2) and the UGB1 (tetravalent PEG, amine-terminated, MW 10,000, Jenkem Technology, USA) were used to prepare covalently crosslinked hydrogels in the following way: GB4 or GB5 and UGB1 were each dissolved in one third of the total volume of the hydrogel mixture in deionized, ultra-pure water with the 3-fold concentration, as shown in Table 1, and additionally treated for 5 min at 4° C. with an ultrasonic bath (RK 100H, Bandelin, Germany). Thereafter, all solutions were tempered at 4° C. for the additional steps: 1-ethyl-3-(3-dimethylaminopropyl) carbodiimide (EDC, Sigma-Aldrich, Germany) and N-hydroxysulfosuccinimides (sulpho-NHS; Sigma-Aldrich, Germany) were also dissolved in one sixth of the total reaction mixture and tempered at 4° C. The molar ratio of UGB1:EDC:sulfoNHS is 1:8:4 (1:2:1 for each amino group of UGB1). In the next step, the EDC and the sulfo-NHS were added to the GB4 or GB5 and mixed by pipetting, and after a wait time of 30 seconds the reaction mixture was mixed on the vortex shaker (VWR, Germany) for 10 seconds, followed by another wait of 20 seconds. After this procedure, with a total activation time of 1 min, the UGB1 on the vortex shaker was pipetted to the activated GB4 or GB5 and then mixed with the vortex shaker for another 10 sec. The final reaction mixture (the concentrations of UGB1 and GB4 or GB5 now correspond to the concentrations given in Table 1) could now be poured by pipetting into arbitrary forms, with the gelation taking place by polymerization over a period of 12 hours. Subsequently, the hydrogels were completely swollen in phosphate buffered saline solution by repeated solution exchange over several hours prior to further use or characterization of the hydrogels.

### Preparation of the Hydrogels for the Cell Culture With Human Endothelial Cells:

For the culture of human endothelial cells, surface-bound gels having a final thickness of about 100 μm were formed by pipetting 11 μl of the final reaction mixture/cm2 onto glass coverslips. The glass coverslips were previously coated with a thin film of poly (ethylene-old-maleic anhydride) according to the procedure of Pompe et. al. Biomacromolecules 2003, 4, 1072-1079 to ensure covalent bonding of the hydrogel. For modification with RGD peptides, the shaped hydrogels swollen in phosphate-buffered saline solution with EDC/sulfo-NHS were dissolved at a concentration of 50 mM EDC and 25 mM sulpho-NHS in 1/15 M phosphate-buffered saline solution at 4° C. and remaining carboxyl groups of GB4 or GB5 were activated for 20 min and then rinsed with borate buffer (100 mM, pH 8.0, 4° C.). Subsequently, the activated hydrogels were reacted with a solution having a concentration of 50 mg/ml of the peptide sequence H2N-GWGGRGDSP-CONH2 (Peptides International, Louisville, KY, USA) dissolved in borate buffer (100 mM, pH 8.0) at room temperature for 2 hours and then washed excessively with phosphate-buffered saline.

### Cell Culture:

Human umbilical vein endothelial cells (HUVECs, Lonza, Germany) were passaged on Promocell C-22010 supplemented with Promocell C-22010 (PromoCell GmbH, Germany) at 37° C. and 5% CO₂ on fibronectin-coated cell culture flasks until reaching 80% confluency. Cells from passage 2 to 6 were used in subsequent experiments.

The RGD-functionalized surface-bound hydrogels were incubated with VEGF-A and FGF-2 at a concentration of 0.565 μg per cm 2 hydrogel surface at RT for 18 hours and then washed 2× with phosphate buffered saline.

50,000 cells/cm² were cultured on the hydrogels functionalized with RGD-and VEGF-165 and FGF-2 in Promocell medium without the supplemental mix. After 24 hours of culturing, the cells were washed, fixed and characterized by light microscopy (Olympus IX73 inverted microscope, Hamburg, Germany). The aspect ratio was determined after measuring individual cells with Fiji image processing software by dividing the cell length by the cell width. The circularity was determined by means of Fiji image processing software according to the following formula: circularity=4·π·(cell area/perimeter)². In each case, 20 cells per image were measured on n=10 independent samples. Statistical evaluation with the GraphPad Prism software and a one-way Anova test revealed significant differences for all conditions studied.

### Preparation of the Hydrogels Based on Poly (4-styrenesulfonic Acid-co-maleic Acid) After Crosslinking Reaction 2 (VN2, See Also Table 1)

Derivatization of Poly (4-styrenesulfonic Acid-co-maleic Acid) With Maleimide Groups:

For the formation of hydrogels according to the crosslinking reaction 2, the GB4 or GB5 must first be functionalized with N-(2-aminoethyl) maleimide trifluoroacetate (Sigma, Germany). For this purpose, 500 mg (25 μmole) of the respective GB4 or GB5 were dissolved in 2.7 ml deionized, ultrapure water and stirred on ice for 10 min. The sample container is a 25 ml snap-cover glass. Subsequently, 65.14 mg of NHS (0.30 mmole) were dissolved in 400 μl and 115.02 mg (0.60 mmole) of EDC dissolved in 200 μl of ice-cold deionized ultrapure water were added to the GB4 or GB5 solution and waited for 5 min. In the next step, 76.25 mg (0.30 mmole) of N-(-2-aminoethyl) maleimide trifluoroacetate dissolved in 200 μl of MilliQ water were added dropwise over 40 seconds after the completed activation time to activated GB4 or GB5. The reaction mixture is stirred on ice for another 10 min. Finally, the ice bath is removed and the mixture is stirred overnight at room temperature. The product is placed in a dialysis tubing with an exclusion size of MWCO=5 kDa. The dialysis takes place over two days; on the first day, the dialysis is performed against 2.5 liters of mono-molar sodium chloride solution for 6 h. Here, the solution is exchanged after every 2 hours. After 6 h, the dialysis is then carried out overnight against deionized, ultrapure water. On the second day, the dialysis is carried out for 8 h against deionized, ultrapure water, the water being exchanged after every 2 hours. In the last step, the solution is freeze-dried.

Size-Exclusion Chromatography for Characterization of GB4/GB5-maleimide Derivatives:

The size-exclusion chromatography (SEC) is used to determine the number of maleimide groups per GB4 or GB5 molecule. For this purpose, the peptide RGD-SP (M=990 g/mole) is coupled to the GB4/GB5-maleimide derivatives in various excesses. The examined molar RGD SP excesses are in this case 6.8, 10 or 12 with respect to the GB4 or GB5. Initially, a calibration is performed by mixing 35 μl of the respective RGD-SP concentration with 35 μl of phosphate-buffered saline solution in a sample vessel.

For this study, a BioSEP-SEC S2000 column from Phenomnex (Germany) used, in which the HPLC system Agilent 1100 (Germany) is inserted. The eluent is phosphate-buffered saline solution at a flow rate of 0.5 ml/min. The injection volume is 50 μl.

At the same elution conditions, the determination of the maleimide groups is also carried out. In each case, 35 μl of an RGD concentration are mixed with 35 μl of the GB4/GB5-maleimide derivatives solution in the sample vessel. The method allows an exact determination of the conversion of maleimide with GB4 or GB5.

Preparation of the Hydrogels Based on Poly (4-styrenesulfonic Acid-co-maleic Acid) Derivatives After Crosslinking Reaction 2 (VN2, See Also Table 1):

GB4/GB5 Maleimide derivatives and four-arm, thiol-terminated PEG (UGB2, Mw=10,000 Da, Jenchem, USA, Table 2) are dissolved in phosphate-buffered saline solution at twice the concentration mentioned in Table 1. Thereafter, equal volumes of both components are pipetted into a micro-reaction vessel and shaken for 10 sec on the vortex shaker. Subsequently, a defined volume of the reaction mixture is removed and placed on a 9 mm diameter glass coverslip coated with Sigma Cote®. Another cover slip, coated with Sigma Cote®, is placed on the drop. The coverslips are removed after 30 minutes and the gel slice is swollen for 12 hours in phosphate buffered saline solution. At high UGB2 concentrations, the pH of the UGB2 can be adjusted to between 5-7 pH with 1M HCl to achieve optimal gelation.

### Production of the Hydrogels for Cell Culture With Human Mesenchymal Stromal Cells in 3D:

Matrix metalloprotease (MMP) cleavable hydrogels were prepared using UGB4 (Table 4, synthesized according to the procedure described by Tsurkan et al., Adv. Mater. 2013, 25 (18), 2606-2610) in place of UGB2 according to the aforementioned method.

For this purpose, the cells were suspended in the GB4 or G B5 derivative, and the hydrogels were formed by mixing following the aforedescribed method, and swollen immediately after 5 min gelation time in phosphate buffered saline solution and cell culture medium.

### Cell Culture:

Mesenchymal stem cells (MSCs, ATCC, isolated from adipose tissue, Germany) were cultured in DMEM with 10% fetal calf serum and 1% penicillin/streptomycin under standard culture conditions (5% CO₂ and 37° C.). MSCs of passages 2 to 4 were used in the experiments.

GB-4 or GB-5 derivatives and UGBs were mixed with 1 mole of SEQ ID NO 26 per mole of GB and dissolved in phosphate buffered saline solution (the concentration of GB4 or GB5 derivatives was 3 times the concentration shown in Table 1). After mixing, the solution was incubated for 10-15 min at 37° C., and a concentrated cell suspension was then mixed in one third of the final gel volume with 6×106 cells/ml extrapolated to the final reaction mixture. UGB4 (3-fold concentration of Table 1) was dissolved in another third of the total gel volume in phosphate-buffered saline solution and added to the mixture of GB4/5 derivatives with cells and intermixed by pipetting. After gelation for 5 min, the aforedescribed cell culture medium was added and the hydrogels were incubated for 24 h at 37° C. as previously described.

PrestoBlue® Test: The PrestoBlue® test is performed after an incubation time of 24 h following culturing of the cells in the hydrogels. To this end, 10% solution of the PrestoBlue® dye dissolved in cell culture medium is added to each hydrogel sample and incubated in microtiter plates for 1 h at 37° C. Subsequently, the fluorescence is measured on the microplate photometer (Tecan Spark®, Germany). The excitation wavelength is 560 nm and the fluorescence was measured at a wavelength of 590 nm. The metabolic activity of the cells was indicated by the relative fluorescence values (RFUs). 10 independent experiments with 4 subject determinations (n=40) were evaluated and significant differences (One Way Anova) were found for all conditions.

### Antibacterial Inhibition Zone Assay:

Hydrogel discs (60 μl) were incubated in 1 ml of 50 μg/ml gentamiycin (Sigma Aldrich, Germany) for 18 hours and then washed twice with phosphate-buffered saline solution. The antimicrobial activity of the gentamiycin-charged hydrogels was determined by an inhibition zone assay. For this purpose, Luria broth (LB) agar (Sigma Aldrich, Munich, Germany) plates were prepared according to the manufacturer's instructions. Escherichia coli K12 DH5 (DSMZ, Germany) and Staphylococcus epidermidis PCI 1200 (ATCC, USA) cultures after 12 h growth were diluted to 0.1 at 600 nm and the optical density (OD) was measured. 250 μl of these bacterial suspensions were applied on the respective plates. A sterile cotton cloth was used to distribute the bacteria evenly. Hydrogels UGB1-GB1 01, UGB2-GB4 20 and UGB2-GB5 23 as well as UGB2-UGB3 26, each charged or uncharged with gentamicin, were placed in the four corners on the AGAR plate and the plates were incubated overnight at 37° C. Thereafter, the inhibition zone was measured with a digital caliper. 10 independent experiments with 3 subject definitions (n=30) were evaluated and significant differences (One Way Anova) were found for all conditions.

Determination of the physical-chemical properties of the hydrogels:

To determine the physical properties of the various hydrogel types, 67 μl of unpolymerized hydrogel solution were in each case polymerized between two 9 mm glass slides (Menzel Gläser, Germany) treated with Sigmacote (Sigma-Aldrich, Germany) for 16 h at room temperature and the resulting gel slices were then removed from the glass slides. The diameter of the gel slices was optically determined with a scanner of the type FLA-3100 (Fujitsu, Japan) (diameter in the unswollen state). Thereafter, the gel slices were swollen in phosphate-buffered saline solution (PBS), 0.9% NaCl buffered to pH 7.4 (Sigma-Aldrich, Germany), for 24 h (physiological conditions) and again measured with the scanner of the FLA type. 3100 (Fujitsu, Japan) (diameter in the swollen state). The swelling of the hydrogels was determined from the determined diameters according to the following equation:

$$Swelling = diameter of the swollen hydrogel \hat{} 3 / diameter of the unswollen hydrogel \hat{} 3 ,$$

$$Swelling = \frac{{( diameter of the swollen hydrogel )}^{3}}{{( diameter of the unswollen hydrogel )}^{3}}$$

### see Table 1.

The reported data are each obtained by averaging (MW) from at least four independent samples. In addition, the standard deviation (SD) was specified.

Furthermore, the storage modulus and loss modulus of the hydrogels were determined by oscillatory rheometry (in kilopascals) using a shear rheometer of the type Ares from TA Instruments United Kingdom. For this purpose, 8 mm slices were punched from hydrogels swollen under physiological conditions (phosphate-buffered saline solution (PBS, 0.9% NaCl buffered to pH 7.4 (Sigma-Aldrich, Germany)) for 24 h and measured in a 9 mm plate-plate measurement setup with increasing frequency of 1-100 rad/s at room temperature with low deformation (2%), and the average value was determined over the entire frequency range (1 measurement value per sample). The reported values are the average values of four independently prepared hydrogel disks and ± of the standard deviation are given. The storage modulus of the four hydrogel types was given in Table 1, and the loss modulus was smaller by several orders of magnitude (data not shown).

Table 1 shows the physical-chemical properties of the hydrogels.

The mesh size of the hydrogels can be derived from the experimentally determined storage modulus based on the rubber elasticity theory using the following formula (Polymer Physics, Michael Rubinstein and Ralph H. Colby, 2006, Oxford University Press, Oxford):

$$ξ = {( \frac{{G}^{′} {N}_{A}}{R T} )}^{- 1 / 3} .$$

wherein G′ is the measured storage modulus, N_{A} is the Avogadro constant, R is the universal gas constant and T is the temperature (in Kelvin).

To determine the binding properties of the different hydrogel types, unpolymerized hydrogel solutions were polymerized for 16 h at room temperature between two 5 mm glass slides treated with Sigmacote (Sigma-Aldrich, Germany) (Menzel Glasses) and subsequently swollen for 24 h in phosphate-buffered saline solution (PBS), 0.9% NaCl buffered to pH 7.4 (Sigma-Aldrich, Germany). The total volume was adjusted accordingly to ensure equal molar quantities of GB1, GB2, GB3, and GB4 in the four hydrogel types (see Table 4). For the binding (sequestration) studies, the respective hydrogel disks were incubated for 24 h in 0.5 ml protein LoBind reaction vessels (Eppendorf Tubes, Germany) with the protein mixture corresponding to Table 3 dissolved in 400 μl PBS with 1% (m/v) bovine serum albumin (Sigma-Aldrich, Germany) and 0.05% (m/v) Proclin 300 (Sigma-Aldrich, Germany) (corresponds to solution after incubation 2). For preparing the protein mixture, ProcartaPlex Standard A, B and C (ebioscience, Germany) was dissolved according to the manufacturer's instructions and supplemented with the proteins DKK1 (manufacturer no: 120-30, Peprotech, Germany), sclerostin (manufacturer no: 1406-ST, Peprotech, Germany) and TGFb1 (manufacturer: 100-21, Peprotech, Germany). The individual concentrations are shown in Table 7 (corresponds to solution before incubation 1).

The solutions 1 and 2 were stored at −80° C. until the protein concentration was measured. To determine the protein concentrations in the solutions 1 and 2, the samples were measured according to the manufacturer's instructions with ProcartaPlex Human Chemokine Panel 1 (ebioscience, Germany) in combination with the corresponding ProcarteaPlex Simplex kits on a device of the type Bioplex 200 (Biorad, Germany).

The binding (sequestration) of the signaling molecules was determined from the determined concentrations of solution 1 and 2 according to the following equation: binding in %=(1 concentration in solution 2 (after incubation)/concentration in solution 1 (before incubation))×100.

Table 4 shows the activation time of the carboxyl groups of GB1 to 4 in min and the gel volume used for the binding studies.

The sulfated or sulfonated hydrogels are characterized by their charge distribution at the charged building block (GB) in mole sulfate or sulfonate groups per mole of polymer, and by the concentration of sulfate or sulfonate groups in the hydrogel volume swollen under physiological conditions in mmole sulfate or sulfonate/ml of hydrogel, and the number of sulfate or sulfonate groups per WE divided by the molar mass of the WE in the specified regions. The calculation was carried out based on the molar concentrations of the hydrogel building blocks during hydrogelation (see Table 1) and the volume swelling (Table 1), assuming that the hydrogel building blocks are quantitatively built into the network.

The concentration of sulfate or sulfonate groups in the unswollen hydrogel was calculated from the concentration of the GBs in the unswollen hydrogel×number of repeat units×number of sulfate or sulfonate groups per repeat unit. The concentration of sulfate or sulfonate in the swollen gel (Table 1) was calculated from the concentration of sulfate or sulfonate groups in the unswollen hydrogel divided by the swelling (see Table 1).

Extraction experiments have not yielded any measurable elution of the gel components, so that the assumption of complete incorporation of the gel components is considered justified.

Table 3 shows the percentage of bound quantities of signaling molecules in the hydrogels normalized to the concentration of soluble factors prior to incubation, as previously discussed. The percentages refer to percent by mass.

The molecular size of the substances, which are also referred to as signal substances or factors, is expressed in kilo daltons (kDa).

The proteins (signaling molecules) are uniquely assigned by way of abbreviations (Table 3) and the UniProt Identification Numbers (Uniprot ID) of the Universal Protein Resource database (UniProt; http://www.uniprot.org/).

The isoelectric point (IEP) and molar mass were determined based on the fully biologically processed amino acid sequence using the program ExPASy ProtParm (http://web.expasy.org/protparam/; reference: Gasteiger, E. et al., The Proteomics Protocols Handbook 571-607 (2005)).

The structural parameters of the proteins were determined using the database ExPASy PROSITE based on the UniProt identification numbers (http://prosite.expasy.org/; Sigrist, C.J.A. et al., New and continuing developments at ExPASy PROSITE; References: (1) Nucleic Acids Res 41, (2013); (2) Sigrist, C.J. A. et al., PROSITE: a documented database using patterns and profiles as motif descriptors., Brief. Bioinform., 3, 265-274 (2002)).

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed is:

1. A covalently crosslinked hydrogel, comprising:
charged building blocks in the form of poly (4-styrenesulfonic acid-co-maleic acid); and
uncharged building blocks in the form of polymers or crosslinking molecules with at least two amino groups or thiol groups,
wherein the charged and uncharged building blocks are crosslinked to form a polymer network by
activating the carboxyl groups of the poly (4-styrenesulfonic acid-co-maleic acid) with 1-ethyl-3-(3-dimethylaminopropyl) carbodiimide (EDC)/N-hydroxysulfosuccinimide (sulfo-NHS), and
a) direct crosslinking of the poly (4-styrenesulfonic acid-co-maleic acid) with the polymers containing the amine groups or the crosslinker molecules having the at least two amino groups each with amide formation, or
b) functionalization of the activated carboxyl groups with bifunctional crosslinker molecules, each having an amino group and a group capable of Michael-type addition, and subsequent crosslinking of the poly (4-styrenesulfonic acid-co-maleic acid) with the polymers containing thiol groups or the crosslinker molecules having the at least two thiol groups each via a respective Michael-type addition.

2. The covalently crosslinked hydrogel of claim 1, wherein the Michael-type addition group is selected from the group consisting of a maleimide group, vinylsulfone group or acrylate group.

3. The covalently crosslinked hydrogel of claim 1, wherein the polymers are selected from the group consisting of polyethylene glycols (PEG), poly (2-oxazolines) (POX), polyvinyl pyrrolidones (PVP), polyvinyl alcohols (PVA) and polyacrylamides (PAM), and wherein the crosslinker molecules containing amine or thiol groups are non-polymeric, bifunctional crosslinker molecules.

4. The covalently crosslinked hydrogel of claim 1, wherein the charged building blocks are poly (4-styrenesulfonic acid-co-maleic acid) with variable molar ratios of 4-styrenesulfonic acid to maleic acid in a range from 6:1 to 1:6 and molar masses in a range from 5,000 to 100,000 g/mole.

5. The covalently crosslinked hydrogel of claim 1, wherein the uncharged building blocks are polymers conjugated with enzymatically cleavable peptides having a peptide sequencer comprising either lysine or cysteine as reactive amino acid.

6. The covalently crosslinked hydrogel of claim 5, wherein the enzymatically cleavable peptides are cleavable by human or bacterial proteases.

7. The covalently crosslinked hydrogel of claim 6, wherein the human or bacterial proteas are selected from the group consisting of MMPs, cathepsins, elastases, aureolysin and blood clotting enzymes or combinations thereof.

8. The covalently crosslinked hydrogel of claim 1, further comprising bioactive and/or antiadhesive molecules having an amino or carboxyl group and/or cell-instructing peptides, said bioactive and/or antiadhesive molecules having a peptide sequence comprising at least one of lysine and cysteine and are bound to the charged building block poly (4-styrenesulfonic acid-co-maleic) acid or to derivatives thereof with Michael-type- addition-groups via the lysine or cysteine forming a covalent bond.

9. The covalently crosslinked hydrogel of claim 8, wherein the bioactive molecules are antimicrobial substances.

10. The covalently crosslinked hydrogel of claim 8, wherein the bioactive molecules are antibiotics or antiseptics, or pharmaceutical active ingredients.

11. The covalently crosslinked hydrogel of claim 8, wherein the anti-adhesive molecules are polyethylene glycols (PEG) or poly (2-oxazolines) (POX).

12. The covalently crosslinked hydrogel of claim 8, wherein the cell-instructing peptides are peptides derived from structural and functional extracellular matrix proteins.

13. The covalently crosslinked hydrogel of claim 12, wherein the cell-instructing peptides are selected from the group consisting of collagen, laminin, tenascin, fibronectin and vitronectin.

14. The covalently crosslinked hydrogel according to claim 11, wherein the bioactive and/or antiadhesive and/or cell-inducible peptides are covalently bonded to the hydrogel networks via enzymatically cleavable peptide sequences.

15. The covalently crosslinked hydrogel of claim 1, wherein the hydrogel material has a storage modulus of 0.2 to 22 kPa.
